(19) United States
(12) Patent Application Publication
(10) Pub. No.: US 2008/0306116 A1
(43) Pub. Date: Dec. 11, 2008

(54) ARYLOXAZOLE, ARYLOXADIAZOLE AND BENZIMIDAZOLE DERIVATIVES

(76) Inventors: Andreas D. Christ, Arlesheim (CH); Rainer E. Martin, Basel (CH); Peter Mohr, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/123,508

(22) Filed: May 20, 2008

(30) Foreign Application Priority Data
Jun. 8, 2007 (EP) 07109846

(51) Int. Cl. A61K 31/454; C07D 405/12

(52) U.S. Cl. 514/326; 546/209

(57) ABSTRACT

This invention relates to compounds of the formula

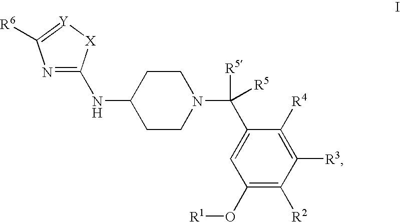

wherein X is O or NR⁸, Y is CR⁷ or N, and R¹ to R⁸ are as defined in the specification, and pharmaceutically acceptable salts thereof. The invention further relates to pharmaceutical compositions containing such compounds, to a process for their preparation and to their use for the treatment and/or prevention of diseases which are associated with the modulation of SST receptors subtype 5.

### PRIORITY TO RELATED APPLICATION(S)

This application claims the benefit of European Patent Application No. 07109846.1, filed Jun. 8, 2007, which is hereby incorporated by reference in its entirety.

### FIELD OF THE INVENTION

The present invention is concerned with novel aryloxazole, aryloxadiazole and benzimidazole derivatives, and their manufacture, pharmaceutical compositions containing them and their use as medicaments. The active compounds of the present invention are useful in the prevention and/or treatment of diabetes mellitus and other disorders.

In particular, the present invention is concerned with compounds of the general formula I

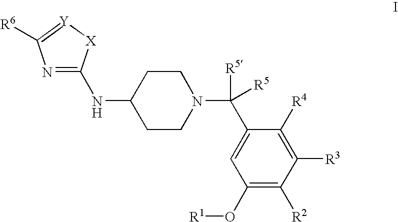

and pharmaceutically acceptable salts thereof.

The compounds of formula I possess pharmaceutical activity, in particular they are modulators of somatostatin receptor activity. More particularly, the compounds are antagonists of the somatostatin receptor subtype 5 (SSTR5).

All documents cited below are expressly incorporated herein by reference.

### BACKGROUND OF THE INVENTION

Diabetes mellitus is a systemic disease characterized by metabolic disorders involving insulin, carbohydrates, fats and proteins, and disorders in the structure and function of blood vessels. The primary symptom of diabetes is hyperglycemia, often accompanied by glucosuria, the presence in urine of large amounts of glucose, and polyuria, the excretion of large volumes of urine. Additional symptoms arise in long-standing diabetes, including degeneration of the walls of blood vessels. Although many different human organs are affected by these vascular changes, the eyes and kidneys appear to be the most susceptible. As such, long-standing diabetes mellitus, even when treated with insulin, is a leading cause of blindness.

There are three recognized types of diabetes mellitus. Type I diabetes or insulin dependent diabetes mellitus (IDDM) is typically of juvenile onset; ketosis develops early in life with much more severe symptoms and has a near-certain prospect of later vascular involvement. Control of Type I diabetes is difficult and requires exogenous insulin administration. Type II diabetes or non-insulin dependent diabetes mellitus (NIDDM) is ketosis-resistant, generally develops later in life, is milder and has a more gradual onset. Gestational diabetes is related to type II diabetes and associated with an increased risk of later development of that disease. Type III diabetes is malnutrition-related diabetes.

NIDDM is a condition that poses a major threat to the health of the citizens of the western world. NIDDM accounts for over 85% of diabetes incidence worldwide and about 160 million people are suffering from NIDDM. The incidence is expected to increase considerably within the next decades, especially in developing countries. NIDDM is associated with morbidity and premature mortality resulting from serious complications, e.g., cardiovascular disease (G. C. Weir and J. L. Leahy, Pathogenesis of non-insulin dependent (Type II) diabetes mellitus, in Joslin's Diabetes Mellitus (Eds. C. R. Kahn and G. C. Weir), 13^{th} Edition, 1994, Lea & Febiger, Malvern, Pa., pp. 240-264). NIDDM is characterized by both fasting and post-prandial hyperglycemia resulting from abnormalities in insulin secretion and insulin action (G. C. Weir et al., vide supra).

The hyperglycemia in patients suffering from NIDDM can usually be initially treated by dieting, but eventually most NIDDM patients have to take oral antidiabetic agents and/or insulin injections to normalize their blood glucose levels. The introduction of orally effective hypoglycemic agents was an important development in the treatment of hyperglycemia by lowering blood glucose levels. Currently, the most widely used oral antidiabetic agents are the sulfonylureas, which act by increasing the secretion of insulin from the pancreas (H. E. Lebovitz, Oral antidiabetic agents, in Joslin's Diabetes Mellitus (Eds. C. R. Kahn and G. C. Weir), 13^{th} Edition, 1994, Lea & Febiger, Malvern, Pa., pp. 508-529), the biguanides (e.g., metformin) which act on the liver and periphery by unknown mechanisms (C. J. Bailey, M. R. C. Path and R. C. Turner N. Engl. J. Med. 1996, 334, 574-579) and the thiazolidinediones (e.g., rosiglitazone/Avandia®), which enhance the effects of insulin at peripheral target sites (G. L. Plosker and D. Faulds Drugs 1999, 57, 409-438). These existing oral therapies which comprise a wide variety of biguanide, sulionylurea and thiazolidinedione derivatives have been used clinically as hypoglycemic agents. However, all three classes of compound have side effects. The biguanides, for example metformin, are unspecific and in certain cases have been associated with lactic acidosis, and need to be given over a longer period of time, i.e. they are not suitable for acute administration (C. J. Bailey et al., vide supra). The sulfonylureas, though having good hypoglycemic activity, require great care during use because they frequently cause serious hypoglycemia and are most effective over a period of circa ten years. The thiazolidinedlones may cause weight gain and deterioration of cardiovascular function following chronic administration (G. L. Plosker and D. Faulds, vide supra) and troglitazone has been associated with the occurrence of serious hepatic dysfunction.

Thus, there is a significant and rising need for antidiabetic drugs that have novel mechanisms of action, thereby avoiding side effects produced by known therapies. The hormone somatostatin (SST) is primarily produced in the intestinal tract and in the pancreas. In addition it acts as a neurotransmitter. The hormone is involved through its receptors in the regulation of several other hormones and in immunoregulation. In particular, SST suppresses the secretion of insulin by pancreatic β cells and the secretion of glucagon-like peptide 1 (GLP-1) by L cells. GLP-1 in turn is one of the most potent stimulators of insulin production and secretion and is a trophic factor for β cells. In addition, GLP-1 directly increases peripheral glucose disposal (e.g., D. A. D'Alessio, S. E. Kahn, C. R. Leusner and J. W. Ensinck, J. Clin. Invest. 1994, 93, 2263-2266). β and L cells express SST receptor subtype 5 (SSTR5) and agonizing this receptor suppresses insulin and GLP-1 secretion in humans and in animal models (e.g., Y. Zambre, Z. Ling, M.-C. Chen, X. Hou, C.-W. Woon, M. Culler, J. E. Taylor, D. H. Coy, C. van Schravendrjk, F. Schuit, D. G. Pipeleers and D. L. Eizirik Biochem. Pharmacol. 1999, 57, 1159-1164; S. P. Fagan, A. Azizzadeh, S. Moldovan, M. K. Ray, T. E. Adrian, X. Ding, D. H. Coy and F. C. Brunicardi Surgery 1998, 124, 254-258; M. Norman, S. Moldovan, V. Seghers, X.-P. Wang, F. J. DeMayo and F. C. Brunicardi Ann. Surg. 2002, 235, 767-774; T. A. Tirone, M. A. Norman, S. Moldovan, F. J. DeMayo, X.-P. Wang, F. C. Brunicardi Pancreas 2003, 26, e67-73; M. Z. Strowski, M. Köhler, H. Y. Chen, M. E. Trumbauer, Z. Li, D. Szalkowski, S. Gopal-Truter, J. K. Fisher, J. M. Schaeffer, A. D. Blake, B. B. Zhang and H. A. Wilkinson Mol. Endocrinol. 2003, 17, 93-106). Exenatide, a GLP-1 mimetic, is available for the treatment of patients with type II diabetes. However, this compound needs to be delivered by subcutaneous injection (e.g., M. A. Nauck, S. Duran, D. Kim, D. Johns, J. Northrup, A. Festa, R. Brodows and M. Trautmann Diabetologia 2007, 50, 259-267).

Consequently, antagonizing the effect of SST would lead to increased peripheral glucose disposal and higher plasma insulin concentrations. Additionally, SSTR5 knockout mice demonstrated higher insulin sensitivity than litternates (M. Z. Strowski, M. Köhler et al., vide supra). In patients suffering from impaired glucose tolerance and NIDDM, these combined effects would moderate the dangerous hyperglycemia and accordingly reduce the risk of tissue damage. If such SSTR5 antagonists are sufficiently selective over the other four SST receptors, little influence is expected on secretion of other hormones. Particularly, selectivity over SST receptor subtype 2 avoids increased glucagon secretion (K. Cejvan, D. H. Coy and S. Efendic Diabetes 2003, 52, 1176-1181; M. Z. Strowski, R. M. Parmar, A. D. Blake and J. M. Schaeffer Endocnnology 2000, 141, 111-117). Advantageous over established therapies is the dual mechanism of action to increase insulin secretion (directly on pancreatic β cells and indirectly through GLP-1 release from L cells) and to increase glucose disposal, whereby SSTR5 antagonists could have the potential to beneficially influence insulin resistance in patients with NIDDM. In summary, SSTR5 antagonists are expected to beneficially influence NIDDM, the underlying impaired fasting glucose and impaired glucose tolerance, as well as complications of long-standing, insufficiently controlled diabetes mellitus.

GLP-1 is known as an endogenous regulator of gastrointestinal motility and of food intake reducing appetite as shown in laboratory animals, healthy volunteers and patients with NIDDM (E. Näslund, B. Barkeling, N. King, M. Gutniak, J. E. Blundell, J. J. Hoist, S. Rössner and P. M. Hellström Int. J. Obes. 1999, 23, 304-311; J.-P. Gutzwiller, B. Göke, J. Drewe, P. Hildebrand, S. Ketterer, D. Handschin, R. Winterhalder, D. Conen and C. Beglinger Gut 1999, 44, 81-88; J.-P. Gutzwiller, J. Drewe, B. Göke, H. Schmidt, B. Rohrer, J. Lareida and C. Beglinger Am. J. Physiol. 1999, 276, R1541-1544; M. D. Turton, D. O'Shea, I. Gunn, S. A. Beak, C. M. Edwards, K. Meeran, S. J. Choi, G. M. Taylor, M. M. Heath, P. D. Lambert, J. P. Wilding, D. M. Smith, M. A. Ghatei, J. Herbert and S. R. Bloom Nature 1996, 379, 69-72; A. Flint, A. Raben, A. Astrup and J. J. Holst J. Clin. Invest. 1998, 101, 515-520; M. B. Toft-Nielsen, S. Madsbad and J. J. Holst Diabetes Care 1999, 22, 1137-1143; P. K. Cheikani, A. C. Haver and R. D. Reidelberger Am. J. Physiol. 2005, 288, R1695-R1706; T. Miki, K. Minami, H. Shinozaki, K. Matsumura, A. Saraya, H. Ikeda, Y. Yamada, J. J. Holst and S. Seino Diabetes 2005, 54, 1056-1063); thus, elevated GLP-1 will also counteract obesity, a typical condition associated with and leading to NIDDM.

GLP-1 further colocalizes with peptide YY (PYY). Thus, PYY could potentially also be increased by SSTR5 antagonists (K. Mortensen, L. L. Lundby and C. Orsov Annals N.Y. Acad. Sci. 2000, 921, 469-472). There is evidence that PYY increases satiety, reduces body weight and improves glycemic control (N. Vrang, A. N. Madsen, C. M. Tang, G. Hansen and P. J. Larsen Am. J. Physiol. Regul. Integr. Comp. Physiol. 2006, 291, R367-R375; A. P. Sileno, G. C. Brandt, B. M. Spann and S. C. Quay Int. J. Obes. Lond. 2006, 30, 68-72; C. J. Small and S. R. Bloom Expert Opin. Investig. Drugs 2005, 14, 647-653). Taken together, SSTR5 antagonists could have the potential to act on obesity also through PYY.

GLP-1 is co-secreted with GLP-2 that is, consequently, also regulated by SST through SSTR5 (L. Hansen, B. Hartmann, T. Bisgaard, H. Mineo, P. N. Jørgensen and J. J. Holst Am. J. Phys. 2000, 278, E1010-1018). GLP-2 is enterotrophic and beneficial in patients with malabsorption of certain origins, such as short bowel syndrome (D. G. Burrin, B. Stoll and X. Guan Domest. Anim. Endocrinol. 2003, 24, 103-122; K. V. Haderslev, P. B. Jeppesen, B. Hartmann, J. Thulesen, H. A. Sorensen, J. Graff, B. S. Hansen, F. Tofteng, S. S. Poulsen, J. L. Madsen, J. J. Holst, M. Staun and P. B. Mortensen Scand. J. Gastroenterol. 2002, 37, 392-398; P. B. Jeppesen J. Nutr. 2003, 133, 3721-3724).

Moreover, there is increasing evidence for a role of SST on immune cells and expression of SSTR5 on activated T lymphocytes (T. Talme, J. Ivanoff, M. Hägglund, R. J. J. van Neerven, A. Ivanoff and K. G. Sundqvist Clin. Exp. Immunol. 2001, 125, 71-79; D. Ferone, P. M. van Hagen, C. Semino, V. A. Dalm, A. Barreca, A. Colao, S. W. J. Lamberts, F. Minuto and L. J. Hofland Dig. Liver Dis. 2004, 36, S68-77; C. E. Ghamrawy, C. Rabourdin-Combe and S. Krantic Peptides 1999, 20, 305-311). Consequently, SSTR5 antagonists could also prove valuable in treating diseases characterized by a disturbed immune system, such as inflammatory bowel disease. It is therefore an object of the present invention to provide selective, directly acting SSTR5 antagonists. Such antagonists are useful as therapeutically active substances, particularly In the treatment and/or prevention of diseases which are associated with the modulation of SST receptors subtype 5.

### SUMMARY OF THE INVENTION

In an embodiment of the present invention, provided is a compound of formula I,

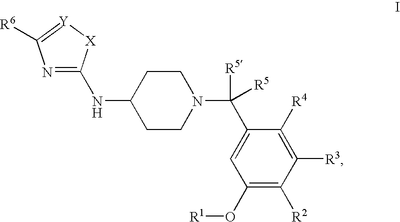

### Wherein:

X is O or NR⁸, wherein R⁸ is hydrogen or C₁₋₇-alkyl;
Y is CR⁷ or N;
R¹ is selected from the group consisting of ethyl, 2-fluoroethyl, isopropyl and isobutyl;
R² is selected from the group consisting of hydrogen, C₁₋₇-alkyl,
   hydroxy, C₁₋₇-alkoxy,
   C₃₋₇-cycloalkyl, —O—C₃₋₇-cycloalkyl,
   halogen, halogen-C₁₋₇-alkyl,
   —C(O)OR⁹, wherein R⁹ is C₁₋₇-alkyl,
   —NH—C(O)—R¹⁰, wherein R¹⁰ is C₁₋₇-alkyl, amino,
   pyridyl, imidazolyl, triazolyl, pyrrolyl, phenyl, and
   phenyl substituted by one to three substituents selected from the group consisting of halogen, halogen-C₁₋₇-alkyl and halogen-C₁₋₇-alkoxy;
R³ is selected from the group consisting of hydrogen, C₁₋₇-alkoxy, amino,
   —NH—C(O)—R¹¹, wherein R¹¹ is C₁₋₇-alkyl,
   —O-benzyl and —O-tetrahydropyranyl;
or R² and R³ are bonded to each other to form a ring together with the carbon atoms they are attached to and R² and R³ together are
   —CH═CH—NH— or —CH═CH—C(CH₃)₂—O—;
R⁴ is selected from the group consisting of hydrogen, halogen, pyridyl and pyrimidyl;
R⁵ and R^{5′} independently from each other are selected from hydrogen or methyl;
one of R⁶ or R⁷ is phenyl or phenyl substItuted by one to three substituents selected from the group consisting of
   halogen, halogen-C₁₋₇-alkyl, halogen-C₁₋₇-alkoxy,
   hydroxy, C₁₋₇-alkoxy, hydroxy-C₁₋₇-alkyl, hydroxy-C₂₋₇-alkoxy,
   di-hydroxy-C₃₋₇-alkoxy, C₁₋₇-alkoxy-C₂₋₇-alkoxy, C₁₋₇-alkoxy-hydroxy-C₃₋₇-alkoxy,
   C₃₋₇-cycloalkyl-C₁₋₇-alkoxy, C₃₋₇-cycloalkoxy,
   C₁₋₇-alkyl, C₃₋₇-cycloalkyl,
   cyano, cyano-C₁₋₇-alkoxy,
   C₁₋₇-alkylamino, di-C₁₋₇-alkylamino, amino-C₂₋₇-alkoxy, amino-C₁₋₇-alkyl,
   C₁₋₇-alkylsulfonyl, —O—C₁₋₇-alkylsulfonyl, C₁₋₇-alkylsulfonyl-C₂₋₇-alkoxy, fluorophenyl, pyridyl, tetrazolyl and tetrazolyl-C₁₋₇-alkoxy,
   —C₁₋₇-alkyl-C(O)NR¹²R¹³ or —O—C₁₋₇-alkyl-C(O)NR¹²R¹³, wherein R¹¹ and R¹² are
   C₁₋₇-alkyl,
   —C₁₋₇-alkyl-C(O)OR¹⁴, wherein R¹⁴ is C₁₋₇-alkyl, and
   —O—C₁₋₇-alkyl-C(O)OR¹⁵, wherein R¹⁵ is C₁₋₇-alkyl,
and the other one of R⁶ or R⁷ is hydrogen or absent in case Y is N;
or R⁶ and R⁷ are bonded to each other to form a ring together with the carbon atoms they are attached to and R⁶ and R⁷ together are —CH═CH—CH═CH—;
and pharmaceutically acceptable salts thereof.

In another embodiment of the present invention, provided is a process for the manufacture of compounds according to formula I, comprising the steps of:
a) reacting a piperidine of the formula

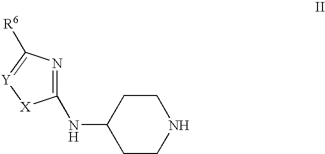

wherein X, Y and R⁶ are as defined above,
with an aldehyde of the formula

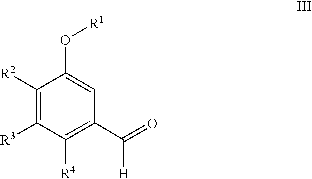

wherein R¹ to R⁴ are as defined above,
by employing a reducing agent to obtain a compound of the formula

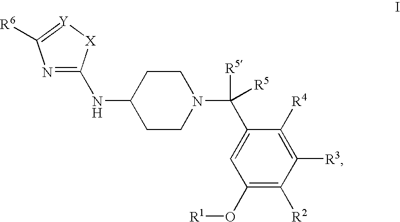

and, if desired, converting the compound of formula I into a pharmaceutically acceptable salt; or, alternatively,
b) alkylating a piperidine of the formula

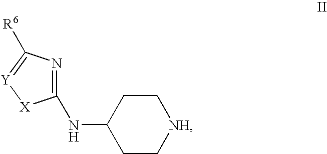

wherein X, Y and R⁶ are as above,
with a compound of the formula

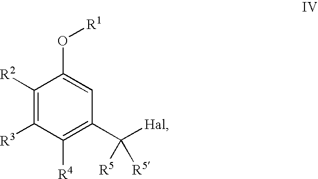

wherein R¹ to R⁵ and R^{5′} are as defined above and Hal means a leaving group, under basic conditions to obtain a compound or formula

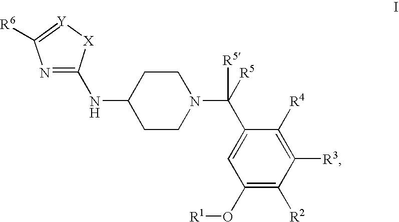

and, if desired, converting the compound of formula I into a pharmaceutically acceptable salt; or, alternatively,
c) coupling an amine of the general formula

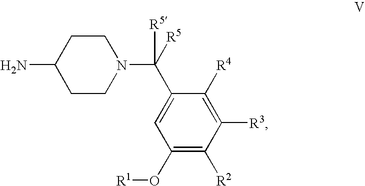

wherein R¹ to R⁵ and R^{5′} are as above,
with a chloride of the formula

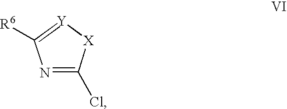

wherein X, Y and R⁶ are as defined herein before, by employing microwave conditions in the presence of a base to obtain a compound of the formula

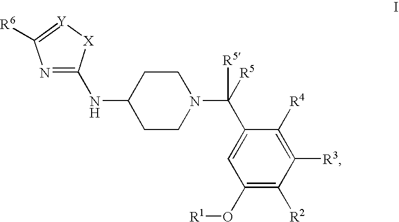

and, if desired, converting the compound of formula I into a pharmaceutically acceptable salt.

In a further embodiment of the present invention, provided is a pharmaceutical composition, comprising a therapeutically effective amount of a compound according to formula I and a pharmaceutically acceptable carrier and/or adjuvant.

### DETAILED DESCRIPTION

In the present description the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “lower alkyl” or “C₁₋₇-alkyl”, alone or in combination, signifies a straight-chain or branched-chain alkyl group with 1 to 7 carbon atoms, preferably a straight or branched-chain alkyl group with 1 to 4 carbon atoms. Examples of straight-chain and branched C₁-C₇ alkyl groups are methyl, ethyl, propyl, isopropyl, butyl, isobutyl, tert-butyl, the isomeric pentyls, the isomeric hexyls and the isomeric heptyls, preferably methyl, ethyl and isopropyl, and most preferred the groups specifically exemplified herein.

The term “cycloalkyl” or “C₃₋₇-cycloalkyl” refers to a monovalent carbocyclic radical of three to seven, preferably three to five carbon atoms. This term is further exemplified by radicals such as cyclopropyl, cyclobutyl, cyclopentyl, cyclohexyl and cycloheptyl, with cyclopentyl being especially preferred.

The term “alkoxy” refers to the group R′—O—, wherein R′ is alkyl. The term “lower alkoxy” or “C₁₋₇-alkoxy” refers to the group R′—O—, wherein R′ is lower alkyl and the term “lower alkyl” has the previously given significance. Examples of lower alkoxy groups are e.g., methoxy, ethoxy, n-propoxy, isopropoxy, n-butoxy, iso-butoxy, sec-butoxy and tert-butoxy, preferably methoxy and ethoxy and most preferred the groups specifically exemplified herein.

The term “cycloalkoxy” or “C₃₋₇-cycloalkoxy” refers to the group R″—O—, wherein R″ is cycloalkyl as defined above. Preferred cycloalkoxy is cyclobutoxy.

The term “lower alkoxyalkyl” or “C₁₋₇-alkoxy-C₁₋₇-alkyl” refers to lower alkyl groups as defined above wherein at least one of the hydrogen atoms of the lower alkyl group is replaced by an alkoxy group as defined above. Among the preferred lower alkoxyalkyl groups are methoxymethyl, methoxyethyl and ethoxymethyl.

The term “lower alkoxyalkoxy” or “C₁₋₇-alkoxy-C₂₋₇-alkoxy” refers to lower alkoxy groups as defined above wherein at least one of the hydrogen atoms of the lower alkoxy group is replaced by an alkoxy group as defined above. Among the preferred lower alkoxyalkoxy groups are 2-methoxy-ethoxy and 3-methoxy-propoxy.

The term “halogen” refers to fluorine, chlorine, bromine and iodine, with fluorine, chlorine and bromine being preferred, and chlorine and bromine being most preferred.

The term “lower halogenalkyl” or “halogen-C₁₋₇-alkyl” refers to lower alkyl groups as defined above wherein at least one of the hydrogen atoms of the lower alkyl group is replaced by a halogen atom, preferably fluoro or chloro, most preferably fluoro. Among the preferred halogenated lower alkyl groups are trifluoromethyl, difluoromethyl, difluoroethyl, fluoromethyl and chloromethyl, with trifluoromethyl and difluoroethyl being especially preferred.

The term “lower halogenalkoxy” or “halogen-C₁₋₇-alkoxy” refers to lower alkoxy groups as defined above wherein at least one of the hydrogen atoms of the lower alkoxy group is replaced by a halogen atom, preferably fluoro or chloro, most preferably fluoro. Among the preferred halogenated lower alkyl groups are trifluoromethoxy, difluoromethoxy, fluoromethoxy and chloromethoxy, with trifluoromethoxy being especially preferred.

The term “lower hydroxyalkyl” or “hydroxy-C₁₋₇-alkyl” refers to lower alkyl groups as defined above wherein at least one of the hydrogen atoms of the lower alkyl group is replaced by a hydroxy group. Examples of lower hydroxyalkyl groups are hydroxymethyl or hydroxyethyl, but also groups having two hydroxy groups such as 1,3-dihydroxy-2-propyl.

The term “lower hydroxyalkoxy” or “hydroxy-C₁₋₇-alkoxy” refers to lower alkoxy groups as defined above wherein at least one of the hydrogen atoms of the lower alkoxy group is replaced by a hydroxy group. Examples of lower hydroxyalkoxy groups are hydroxymethoxy or hydroxyethoxy.

The term “lower dihydroxyalkoxy” or “di-hydroxy-C₃₋₇-alkoxy” refers to lower alkoxy groups as defined above wherein at least two of the hydrogen atoms of the lower alkoxy group are replaced by a hydroxy group. An example of a lower dihydroxyalkoxy group is 2,3-dihydroxy-propyl-1-oxy.

The term “lower alkoxy-hydroxy-alkoxy” or “C₁₋₇-alkoxy-hydroxy-C₃₋₇-alkoxy” refers to lower alkoxy groups as defined above wherein one of the hydrogen atoms of the lower alkoxy group is replaced by an alkoxy group as defined above and one of the hydrogen atoms is replaced by a hydroxy group. An example of a lower alkoxy-hydroxy-alkoxy group is 2-hydroxy-3-methoxy-propyl-1-oxy.

The term “lower alkylsulfonyl” or “C₁₋₇-alkylsulfonyl” refers to the group —S(O)₂—R′, wherein R′ is a lower alkyl group as defined herein before. Examples of lower alkylsulfonyl groups are methylsulfonyl or ethylsulfonyl.

The term “lower cyanoalkoxy” or “cyano-C₁₋₇-alkoxy” refers to lower alkoxy groups as defined above wherein at least one of the hydrogen atoms of the lower alkoxy group is replaced by a cyano group. A preferred lower cyanoalkoxy group is cyanomethoxy.

The term “triazolyl” means a group selected from 1H-[1,2,4]triazolyl, 4H-[1,2,4]triazolyl, 1H-[1,2,3]triazolyl, 2H-[1,2,3]triazolyl and 4H-[1,2,3]triazolyl. Preferred is 1H-[1,2,4]triazolyl.

The term amino refers to the group —NH₂.

The term “lower aminoalkoxy” or “amino-C₂₋₇-alkoxy” refers to lower alkoxy groups as defined above wherein at least one of the hydrogen atoms of the lower alkoxy group is replaced by an amino group. A preferred lower aminoalkoxy group is 2-amino-ethoxy.

The term “alkylamino” or “C₁₋₇-alkylamino” refers to the group —NHR′, wherein R′ is lower alkyl and the term “lower alkyl” has the previously given significance. A preferred alkylamino group is methylamino.

The term “dialkylamino” or “di-C₁₋₇-alkylamino⇄ refers to the group —NR′R″, wherein R′ and R″ are lower alkyl and the term “lower alkyl” has the previously given significance. A preferred dialkylamino group is dimethylamino.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxylic acid, maleic acid, malonic acid, salicylic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like. The compound of formula I can also be present in the form of zwitterions. Particularly preferred pharmaceutically acceptable salts of compounds of formula I are the hydrochloride salts.

The compounds of formula I can also be solvated, e.g., hydrated. The solvation can be effected in the course of the manufacturing process or can take place, e.g., as a consequence of hygroscopic properties of an initially anhydrous compound of formula I (hydration). The term “pharmaceutically acceptable salts” also includes physiologically acceptable solvates.

“Isomers” are compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center”.

In detail, the present invention relates to compounds of the general formula I

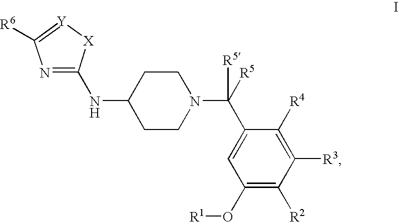

wherein
X is O or NR⁸, wherein R⁸ is hydrogen or C₁₋₇-alkyl;
Y is CR⁷ or N;
R¹ is selected from the group consisting of ethyl, 2-fluoroethyl, isopropyl and isobutyl;
R² is selected from the group consisting of hydrogen, C₁₋₇-alkyl,
   hydroxy, C₁₋₇-alkoxy,
   C₃₋₇-cycloalkyl, —O—C₃₋₇-cycloalkyl,
   halogen, halogen-C₁₋₇-alkyl,
   —C(O)OR⁹, wherein R⁹ is C₁₋₇-alkyl,
   —NH—C(O)—R¹⁰, wherein R¹⁰ is C₁₋₇-alkyl, amino,
   pyridyl, imidazolyl, triazolyl, pyrrolyl, phenyl, and
   phenyl substituted by one to three substituents selected from the group consisting of halogen, halogen-C₁₋₇alkyl and halogen-C₁₋₇-alkoxy;
R³ is selected from the group consisting of hydrogen, C₁₋₇-alkoxy, amino,
   —NH—C(O)—R¹¹, wherein R¹¹ is C₁₋₇-alkyl,
   —O-benzyl and —O-tetrahydropyranyl;
or R² and R³ are bonded to each other to form a ring together with the carbon atoms they are attached to and R² and R³ together are
   —CH═CH—NH— or —CH═CH—C(CH₃)₂—O—;
R⁴ is selected from the group consisting of hydrogen, halogen, pyridyl and pyrimidyl;
R⁵ and R5′ independently from each other are selected from hydrogen or methyl;
one of R⁶ or R⁷ is phenyl or phenyl substituted by one to three substituents selected from the group consisting of
   halogen, halogen-C₁₋₇-alkyl, halogen-C₁₋₇-alkoxy,
   hydroxy, C₁₋₇-alkoxy, hydroxy-C₁₋₇-alkyl, hydroxy-C₂₋₇-alkoxy,
   di-hydroxy-C₃₋₇-alkoxy, C₁₋₇-alkoxy-C₂₋₇-alkoxy,
   C₁₋₇-alkoxy-hydroxy-C₃₋₇-alkoxy, C₃₋₇-cycloalkyl-C₁₋₇-alkoxy,
   C₃₋₇-cycloalkoxy, C₁₋₇-alkyl, C₃₋₇-cycloalkyl, cyano, cyano-C₁₋₇-alkoxy,
   C₁₋₇-alkylamino, di-C₁₋₇-alkylamino, amino-C₂₋₇-alkoxy, amino-C₁₋₇-alkyl,
   C₁₋₇-alkylsulfonyl, —O—C₁₋₇-alkylsulfonyl, C₁₋₇-alkylsulfonyl-C₂₋₇-alkoxy, fluorophenyl, pyridyl, tetrazolyl and tetrazolyl-C₁₋₇-alkoxy,
   —C₁₋₇-alkyl-C(O)NR¹²R¹³ or —O—C₁₋₇-alkyl-C(O)NR¹²R¹³, wherein R¹¹ and R¹² are
   C₁₋₇-alkyl,
   —C₁₋₇alkyl-C(O)OR¹⁴, wherein R¹⁴ is C₁₋₇-alkyl, and
   —O—C₁₋₇alkyl-C(O)OR¹⁵, wherein R¹⁵ is C₁₋₇-alkyl,
      and the other one of R⁶ or R⁷ is hydrogen or absent in case Y is N; or R⁶ and R⁷ are bonded to each other to form a ring together with the carbon atoms they are attached to and R⁶ and R⁷ together are —CH═CH—CH═CH—; and pharmaceutically acceptable salts thereof.

Preferred compounds of formula I of the present invention are those, wherein R¹ is ethyl or isopropyl.

Furthermore, compounds of formula I according to the invention are preferred, wherein R² is selected from the group consisting of hydrogen, C₁₋₇-alkyl, hydroxy, C₁₋₇-alkoxy, C₃₋₇-cycloalkyl, —O—C₃₋₇-cycloalkyl, halogen, halogen-C₁₋₇-alkyl, amino, phenyl and phenyl substituted by one to three substituents selected from the group consisting of halogen, halogen-C₁₋₇-alkyl and halogen-C₁₋₇-alkoxy.

More preferably, R² is selected from the group consisting of hydrogen, C₁₋₇-alkyl, hydroxy, C₁₋₇-alkoxy, halogen, amino and phenyl substituted by one to three substituents selected from the group consisting of halogen, halogen-C₁₋₇-alkyl and halogen-C₁₋₇-alkoxy. Especially preferred are compounds of formula I, wherein R² is selected from the group consisting of halogen, amino and phenyl substituted by halogen, with those compounds of formula I, wherein R² is halogen, being most preferred.

Preferred are also compounds of formula I according to the present invention, wherein R³ is hydrogen or C₁₋₇-alkoxy. Especially preferred are those compounds of formula I, wherein R³ is C₁₋₇-alkoxy.

Further preferred compounds of formula I according to the invention are those, wherein R² and R³ are bonded to each other to form a ring together with the carbon atoms they are attached to and R² and R³ together are —CH═CH—NH— or —CH═CH—C(CH₃)₂—O—, with those compounds, wherein R² and R³ together are —CH═CH—C(CH₃)₂—O—, being more preferred.

Also preferred are compounds of formula I according to the invention, wherein R⁴ is hydrogen.

Preferred compounds of formula I according to the invention are further those, wherein R⁵ and R5′ are hydrogen.

One group of preferred compounds of formula I according to the invention are also those, wherein X is O and Y is N. These are compounds of the formula Ia.

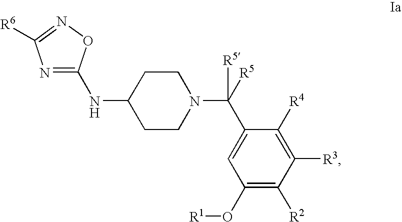

Within this group, compounds of formula I are preferred, wherein R⁶ is phenyl or phenyl substituted by one to three substituents selected from the group consisting of halogen, halogen-C₁₋₇-alkyl, halogen-C₁₋₇alkoxy, hydroxy, C₁₋₇-alkoxy, hydroxy-C₁₋₇-alkyl, hydroxy-C₂₋₇-alkoxy, di-hydroxy-C₃₋₇-alkoxy, C₁₋₇-alkoxy-C₂₋₇-alkoxy, C₁₋₇-alkoxy-hydroxy-C₃₋₇-alkoxy, C₃₋₇-cycloalkyl-C₁₋₇-alkoxy, C₃₋₇-cycloalkoxy, C₁₋₇-alkyl, C₃₋₇-cycloalkyl, cyano, cyano-C₁₋₇-alkoxy, C₁₋₇-alkylamino, di-C₁₋₇-alkylamino, amino-C₂₋₇-alkoxy, amino-C₁₋₇-alkyl, C₁₋₇-alkylsulfonyl, —O—C₁₋₇-alkylsulfonyl, C₁₋₇alkylsulfonyl-C₂₋₇-alkoxy, fluorophenyl, pyridyl, tetrazolyl and tetrazolyl-C₁₋₇-alkoxy, —C₁₋₇alkyl-C(O)NR¹²R¹³ or —O—C₁₋₇-alkyl-C(O)NR¹²R¹³, wherein R¹¹ and R¹² are C₁₋₇-alkyl, —C₁₋₇-alkyl-C(O)OR¹⁴, wherein R¹⁴ is C₁₋₇-alkyl, and —O—C₁₋₇-alkyl-C(O)OR¹⁵, wherein R¹⁵ is C₁₋₇-alkyl.

Especially preferred are those compounds of formula I, wherein R⁶ is phenyl or phenyl substituted by one to three substituents selected from the group consisting of halogen, halogen-C₁₋₇alkyl and C₁₋₇-alkoxy.

Another group of preferred compounds of formula I according to the invention are those, wherein X is NR⁸ and Y is CR⁷. More preferably, these are compounds of formula I, wherein R⁸ is hydrogen or C₁₋₇-alkyl and wherein R⁶ and R⁷ are bonded to each other to form a ring together with the carbon atoms they are attached to and R⁵ and R⁷ together are —CH═CH—CH═CH—. Thus, these are compounds of formula Ib.

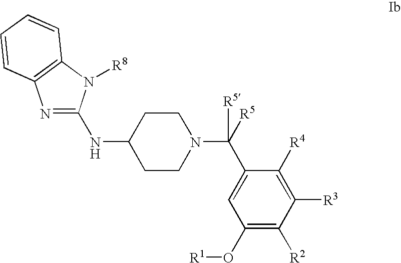

A further group of preferred compounds of formula I according to the present invention are those, wherein X is O and Y is CR⁷. These are compounds of the formula Ic.

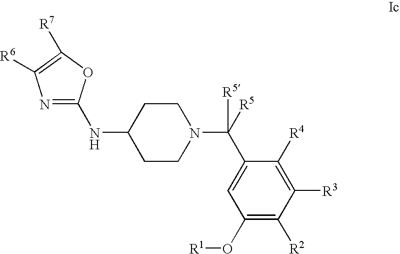

Within this group, those compounds of formula I are preferred, wherein one of R⁶ or R⁷ is phenyl or phenyl substituted by one to three substituents selected from the group consisting of halogen, halogen-C₁₋₇-alkyl, halogen-C₁₋₇-alkoxy, hydroxy, C₁₋₇-alkoxy, hydroxy-C₁₋₇alkyl, hydroxy-C₂₋₇-alkoxy, di-hydroxy-C₃₋₇-alkoxy, C₁₋₇-alkoxy-C₂₋₇-alkoxy, C₁₋₇-alkoxy-hydroxy-C₃₋₇-alkoxy, C₃₋₇-cycloalkyl-C₁₋₇-alkoxy, C₃₋₇-cycloalkoxy, C₁₋₇-alkyl, C₃₋₇-cycloalkyl, cyano, cyano-C₁₋₇alkoxy, C₁₋₇-alkylamino, di-C₁₋₇-alkyl-amino, amino-C₂₋₇-alkoxy, amino-C₁₋₇-alkyl, C₁₋₇-alkylsulfonyl, —O—C₁₋₇-alkylsulfonyl, C₁₋₇-alkylsulfonyl-C₂₋₇-alkoxy, fluorophenyl, pyridyl, tetrazolyl and tetrazolyl-C₁₋₇-alkoxy, —C₁₋₇-alkyl-C(O)NR¹²R¹³ or —O—C₁₋₇-alkyl-C(O)NR¹²R¹³, wherein R¹¹ and R¹² are C₁₋₇-alkyl, —C₁₋₇-alkyl-C(O)OR¹⁴, wherein R¹⁴ is C₁₋₇-alkyl, and —O—C₁₋₇-alkyl-C(O)OR¹⁵, wherein R¹⁵ is C₁₋₇-alkyl,

and the other one of R⁶ or R⁷ is hydrogen.

Especially preferred with this group are compounds of formula I, wherein R⁷ is hydrogen and R⁶ is phenyl or phenyl substituted by one to three substituents selected from the group consisting of halogen, halogen-C₁₋₇-alkyl and C₁₋₇-alkoxy.

Also especially preferred within this group are compounds of formula I, wherein R⁶ is hydrogen and R⁷ is phenyl or phenyl substituted by one to three substituents selected from the group consisting of halogen, halogen-C₁₋₇-alkyl and C₁₋₇-alkoxy.

Examples of preferred compounds of formula I are the following:
[1-(3-ethoxy-4-methyl-benzyl)-piperidin-4-yl]-(3-phenyl-[1,2,4]oxadiazol-5-yl)-amine,
[1-(4-chloro-3-ethoxy-benzyl)-piperidin-4-yl]-(3-phenyl-[1,2,4]oxadiazol-5-yl)-amine,
2-ethoxy-4-[4-(3-phenyl-[1,2,4]oxadiazol-5-ylamino)-piperidin-1-ylmethyl]-phenol,
[1-(3-ethoxy-4-methoxy-benzyl)-piperidin-4-yl]-(3-phenyl-[1,2,4]oxadiazol-5-yl)-amine,
[1-(3-isobutoxy-4-methoxy-benzyl)-piperidin-4-yl]-(3-phenyl-[1 ,2,4]oxadiazol-5-yl)-amine,
{1-[3-(2-fluoro-ethoxy)-4-methoxy-benzyl]-piperidin-4-yl}-(3-phenyl-[1,2,4]oxadiazol-5-yl)-amine,
1-(8-ethoxy-2,2-dimethyl-2H-chromen-6-ylmethyl)-piperidin-4-yl]-(3-phenyl-[1,2,4]oxadiazol-5-yl)-amine,
[1-(3,5-diethoxy4-fluoro-benzyl)-piperidin-4-yl]-(3-phenyl-[1,2,4]oxadiazol-5-yl)-amine,
[1-(4-amino-3,5-diethoxy-benzyl)-piperidin-4-yl]-(3-phenyl-[1,2,4]oxadiazol-5-yl)-amine,
(1H-benzoimidazol-2-yl)-{1-[3-ethoxy-4(1-ethyl-propoxy)-benzyl]-piperidin-4-yl}-amine,
[1-(4-chloro-3-ethoxy-benzyl)-piperidin-4-yl]-(1-methyl-1H-benzoimidazol-2-yl)-amine,
[1-(3-ethoxy-4-methoxy-benzyl)-piperidin-4-yl]-(1-methyl-1H-benzoimidazol-2-yl)-amine,
[1-(3,5-diisopropoxy-benzyl)-piperidin-4-yl]-(1-methyl-1H-benzoimidazol-2-yl)-amine,
[1-(3,5-diethoxy-4-fluoro-benzyl)-piperidin-4-yl]-(1-methyl-1H-benzoimidazol-2-yl)-amine,
[1-(2,6-diethoxy-4′-fluoro-biphenyl-4-ylmethyl)-pipeddin-4-yl]-[4-(3-methoxy-phenyl)-oxazol-2-yl]-amine,
[1-(3,5-diethoxy-4-fluoro-benzyl)-piperidin4-yl]-[4-(3-methoxy-phenyl)-oxazol-2-yl]-amine,
[1-(2,6-diethoxy-4′-fluoro-biphenyl-4-ylmethyl)-piperidin-4-yl]-[4-(4-fluoro-phenyl)-oxazol-2-yl]-amine,
[1-(2-ethoxy-4′-fluoro-biphenyl-4-ylmethyl)-piperidin-4-yl]-[4-(4-fluoro-phenyi)-oxazol-2-yl]-amine,
[1-(3,5-diethoxy-4-fluoro-benzyl)-piperidin-4-yi]-[4(4-fluoro-phenyl)-oxazol-2-yl]-amine,
[1-(4-chloro-3,5-diethoxy-benzyl)-piperidin-4-yl]-[4-(3-methoxy-phenyl)-oxazol-2-yl]-amine,
[1-(3-ethoxy-4-methyl-benzyl)-piperidin-4-yl]-[4-(3-methoxy-phenyl)-oxazol-2-yl]-amine,
[1-(3,5-diisopropoxy-benzyl)-piperidin-4-yl]-[4-(4-fluoro-phenyl)-oxazol-2-yl]-amine,
[1-(4-chloro-3,5-dielhoxy-benzyl)-piperidin-4-yl]-[4-(4-fluoro-phenyl)-oxazol-2-yl]-amine,
[1-(2,6-diethoxy-4′-fluoro-biphenyl-4-ylmethyl)-piparidin-4-yl]-(4-pheny-oxazol-2-yl)-amine,
[1-(4-chloro-3,5-diethoxy-benzyl)-piperidin-4-yl]-(4-phenyl-oxazol-2-yl)-amine,
[1-(3,5-diisopropoxy-benzyl)-piperidin-4-yl]-(4-phenyl-oxazol-2-yl)-amine,
[1-(4-chloro-3,5-diethoxy-benzyl)-piperidin-4-yl]-[4-(4-chloro-phenyl)-oxazo1-2-yl]-amine,
[4-(4-chloro-phenyl)-oxazol-2-yl]-[1-(2,6-diethoxy-4′-fluoro-biphenyl4-ylmethyl)-piperidin-4-yl]-amine,
[4-(4chloro-phenyl)-oxazol-2-yl]-[1-(3,5-diethoxy4-fluoro-benzyl)-piperidin-4-yl]-amine,
[1-(3,5-diethoxy-4-fluoro-benzyl)-piperidin-4-yl]-(4-phenyl-oxazol-2-yl)-amine,
[1-(2-ethoxy-4′-fluoro-biphenyl-4-ylmethyl)-piperidin-4-yl]-[5-(4-fluoro-phenyl)-oxazol-2-yl]-amine,
[1-(2,6-dielhoxy-4′-fluoro-biphenyl-4-ylmethyl)-piperidin-4-yl]-[5-(4-fluoro-phenyl)-oxazol-2-yl]-amine,
[1-(2,6-diethoxy-4′-fluoro-biphenyl-4-ylmethyl)-piperidin-4-yl]-(5-phenyl-oxazol-2-yi)-amine,
[1-(3,5-diethoxy-4-fluoro-benzyl)-piperidin-4-yl]-(5-phenyl-oxazol-2-yl)-amine,
[1-(3,5-diisopropoxy-benzyl)-piperidin-4-yl]-[5-(4-fluoro-phenyl)-oxazol-2-yl]-amine,
[1-(3,5-diethoxy-4-fluoro-benzyl)-piperidin-4-yl]-[5-(4-fluoro-phenyl)-oxazol-2-yl)-amine,
[1-(4-chloro-3,5-diethoxy-benzyl)-piperidin-4-yl]-(5-phenyl-oxazol-2-yl)-amine,
[1-(3-ethoxy-4-methyl-benzyl)-piperidin-4-yl]-(5-phenyl-oxazol-2-yl)-amine,
[1-(4-chloro-3,5-diethoxy-benzyl)-piperidin-4-yl]-[5-(4-fluoro-phenyl)-oxazol-2-yl]-amine,
[1-(2,6-diethoxy-4′-fluoro-biphenyl-4-ylmethyl)-piperidin-4-yl]-[5-(4-methoxy-phenyl)-oxazol-2-yl]-amine,
[1-(4-chloro-3,5-diethoxy-benzyl)-piperidin-4-yl]-[5-(4-methoxy-phenyl)-oxazol-2-yl]-amine,
[1-(3-ethoxy-4-methyl-benzyl)-piperidin-4-yl]-[5-(4-methoxy-phenyl)-oxazol-2-yl]-amine,
[1-(3,5-dilsopropoxy-benzyl)-piperidin-4-yl]-[5-(4-methoxy-phenyl)-oxazol-2-yl]-amine,
[1-(3,5-diethoxy-4-fluoro-benzyl)-piperidin-4-yl]-[5-(4-methoxy-phenyl)-oxazol-2-yl]-amine,
[1-(3-ethoxy-4-methyl-benzyl)-piperidin-4-yl]-[5-(4-fluoro-phenyl)-oxazol-2-yl]-amine,
[1-(3,5-diethoxy-4-methyl-benzyl)-piperidin-4-yl]-(5-phenyl-oxazol-2-yl)-amine,
[4-(4-chloro-phenyl)-oxazol-2-yl]-[1-(3-ethoxy-4-methyl-benzyl)-piperidin-4-yl]-amine,
[1-(3-ethoxy-4-methyl-benzyl)-piperidin-4-yl]-[4-(4-fluoro-phenyl)-oxazol-2-yl]-amine,
[1-(2,6-diethoxy4′-fluoro-biphenyl-4-ylmethyl)-piperidin-4-yl]-[4-(4-trifluoromethyl-phenyl)-oxazol-2-yl]-amine,
and pharmaceutically acceptable salts thereof.

Especially preferred are the following compounds of formula I of the present invention:
[1-(4-amino-3,5-diethoxy-benzyl)-piperidin-4-yl]-(3-phenyl-[1,2,4]oxadiazol-5-yl)-amine,
[1-(3,5-diisopropoxy-benzyl)-piperidin-4-yl]-(1-methyl-1H-benzoimidazol-2-yl)-amine,
[1-(3,5-diethoxy-4-fluoro-benzyl)-piperidin-4-yl]-(1-methyl-1H-benzoimidazol-2-yl)-amine,
[1-(2,6-diethoxy-4′-fluoro-biphenyl4-ylmethyl)-piperidin4-yl]-[4-(4-fluoro-phenyl)-oxazol-2-yl]-amine,
[1-(3,5-diethoxy-4-fluoro-benzyl)-piperidin-4-yl]-[4-(4-fluoro-phenyl)-oxazol-2-yl]-amine,
[1-(4-chloro-3,5-diethoxy-benzyl)-piperidin-4-yl]-(4phenyl-oxazol-2-yl)-amine,
[1-(4-chloro-3,5-diethoxy-benzyl)-piperidin-4-yl]-[4-(4-chloro-phenyl)-oxazol-2-yl]-amine,
[1-(3,5-diethoxy-4-fluoro-benzyl)-piperidin-4-yl]-(4-phenyl-oxazol-2-yl)-amine,
[1-(4-chloro-3,5-diethoxy-benzyl)-piperidin-4-yl]-(5-phenyl-oxazol-2-yl)-amine,
and pharmaceutically acceptable salts thereof.

Furthermore, the pharmaceutically acceptable salts of the compounds of formula I individually constitute preferred embodiments of the present invention.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbens or eluant). The invention embraces all of these forms.

It will be appreciated that the compounds of general formula I in this invention may be derivatized at functional groups to provide derivatives which are capable of conversion back to the parent compound in vivo. Physiologically acceptable and metabolically labile derivatives which are capable of producing the parent compounds of general formula I in vivo are also within the scope of this invention.

A further aspect of the present invention is the process for the manufacture of compounds of formula I as defined above, which process comprises
a) reacting a piperidine of the formula

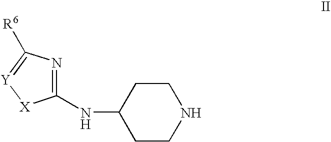

wherein X, Y and R⁶ are as defined herein before,
with an aldehyde of the formula

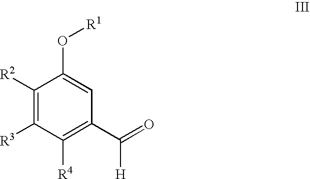

wherein R¹ to R⁴ are as defined herein before,
by employing a reducing agent to obtain a compound of the formula

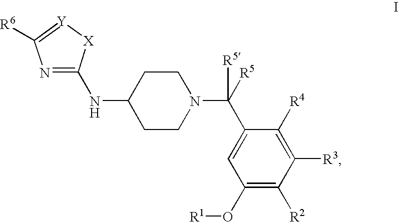

and, if desired, converting the compound of formula I into a pharmaceutically acceptable salt; or, alternatively,
b) alkylating a piperidine of the formula

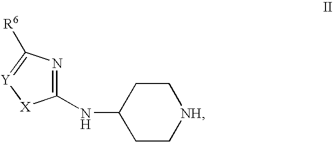

wherein X, Y and R⁶ are as defined herein before,
with a compound of the formula

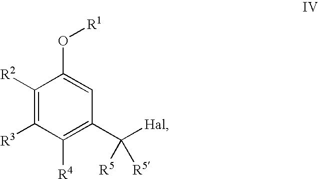

wherein R¹ to R⁵ and R⁵′ are as defined herein before and Hal means a leaving group,
under basic conditions to obtain a compound or formula

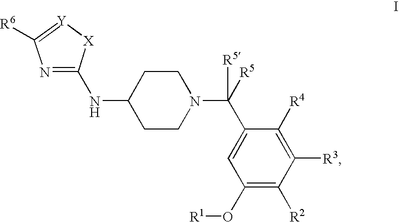

and, if desired, converting the compound of formula I into a pharmaceutically acceptable salt; or, alternatively,
c) coupling an amine of the general formula

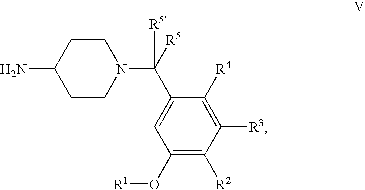

wherein R¹ to R⁵ and R^{5′} are as defined herein before,
with a chloride of the formula

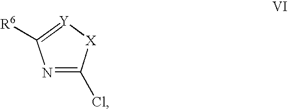

wherein X, Y and R⁵ are as defined herein before, by employing microwave conditions in the presence of a base to obtain a compound of the formula

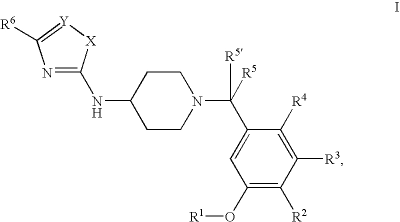

and, if desired, converting the compound of formula I into a pharmaceutically acceptable salt.

The invention further relates to compounds of formula I as defined above, when manufactured according to a process as defined herein before.

Suitable reducing agents are preferably selected from the group consisting of pyridine-BH₃ complex, NaBH(OAc)₃ and NaCNBH₃. The reaction can be carried out under acidic conditions by using a Broensted acid such as acetic acid or formic acid or a Lewis acid (e.g., Ti(iPrO)₄, ZnCl₂) or under buffered conditions (e.g., in the presence of acetic acid and a tertiary amine like N-ethyldiisopropylamine or triethylamine) in a suitable solvent such as dichloromethane, dichloroethane, ethanol or isopropanol (or mixtures thereof) at ambient or elevated temperatures using conventional heating or heating by microwave irradiation.

Suitable leaving groups Hal are preferably halides, but also mesylates or tosylates or alcohols transformed into another leaving group. Preferred leaving groups are selected from the group consisting of iodide, bromide, methanesultonate and chloride.

Diseases which are associated with the modulation of SST receptors subtype 5 are such diseases as diabetes mellitus, particularly type II diabetes mellitus, impaired fasting glucose, impaired glucose tolerance, micro- and macrovascular diabetic complications, post transplantation diabetes mellitus in patients having type I diabetes mellitus, gestational diabetes, obesity, inflammatory bowel diseases such as Crohn's disease or ulcerative colitis, malabsorption, autoimmune diseases such as rheumatoid arthritis, osteoarthritis, psoriasis and other skin disorders, and immunodeficiences. Microvascular diabetic complications include diabetic nephropathy and diabetic retinopathy, whereas macrovascular diabetes-associated complications lead to an increased risk for myocardial infarction, stroke and limb amputation.

The use as medicament for the treatment and/or prevention of diabetes mellitus, particularly type II diabetes mellitus, impaired fasting glucose or impaired glucose tolerance is preferred.

The invention therefore also relates to pharmaceutical compositions comprising a compound as defined above and a pharmaceutically acceptable carrier and/or adjuvant.

Further, the invention relates to compounds as defined above for use as therapeutically active substances, particularly as therapeutic active substances for the treatment and/or prevention of diseases which are associated with the modulation of SST receptors subtype 5.

In another embodiment, the invention relates to a method for the treatment and/or prevention of diseases which are associated with the modulation of SST receptors subtype 5, which method comprises administering a compound of formula I to a human or animal. The method for the treatment and/or prevention of diabetes mellitus, particularly type II diabetes mellitus, impaired fasting glucose or impaired glucose tolerance, is most preferred.

The invention further relates to the use of compounds as defined above for the treatment and/or prevention of diseases which are associated with the modulation of SST receptors subtype 5.

In addition, the invention relates to the use of compounds as defined above for the preparation of medicaments for the treatment and/or prevention of diseases which are associated with the modulation of SST receptors subtype 5. Preferred examples of such diseases are diabetes mellitus, particularly type II diabetes mellitus, impaired fasting glucose or impaired glucose tolerance.

The compounds of formula I can be manufactured by the methods given below, by the methods given in the examples or by analogous methods. Appropriate reaction conditions for the individual reaction steps are known to a person skilled in the art. Starting materials are either commercially available or can be prepared by methods analogous to the methods given below, by methods described in references cited in the text or in the examples, or by methods known in the art.

The synthesis of compounds of the general formula I, can be accomplished according to Scheme 1 or Scheme 2, respectively. Methods for the synthesis of the intermediate aldehydes III are described below in Scheme 3 and Scheme 4, and for the chloro-oxazoles 2 in Scheme 5 and 6, and for the 3-aryl-[1,2,4]oxadiazoles in scheme 7, respectively.

A suitably protected form of 4-amino-piperidine 1 (P means protecting group), e.g., protected by benzyl or as tert-butyl-derivative, or as ethyl carbamate (see “Protective Groups in Organic Synthesis”, T. W. Greene, Wiley-lnterscience, 1999) can first be coupled with a hereoarylchloride of formula 2 by employing microwave conditions under elevated pressure in the presence of a suitable base, e. g., iPrNEt₂ (DIPEA), typically in a solvent such as acetonitrile at elevated temperature, typically 185° C. (Scheme 1, step a). The protecting group can then be removed (step b) by, depending on the group used, e.g., hydrogenation or acid treatment, e. g., HCl or trifluoroacetic acid (see “Protective Groups in Organic Synthesis” above). The liberated amine II can then be alkylated to afford the desired compounds I (step c): 1. by reaction with an aldehyde III under reductive amination conditions (employing a suitable reducing agent such as py-BH₃ complex, NaBH(OAc)₃, NaCNBH₃ under acidic (e.g., acetic acid or Ti(iPrO)₄ or ZnCl₂ as additive) or under basic conditions (no additive) in a solvent such as dichloromethane (DCM), dichloroethane (DCE), ethanol, isopropanol or mixtures thereof at ambient or elevated temperature, or 2. by direct alkylation with alkyl halides IV in solvents such as DMF or DCE at ambient or elevated temperature in the presence of a suitable tertiary amine base (e.g., Et₃N, iPrNEt₂) or inorganic base (e.g. K₂CO₃).

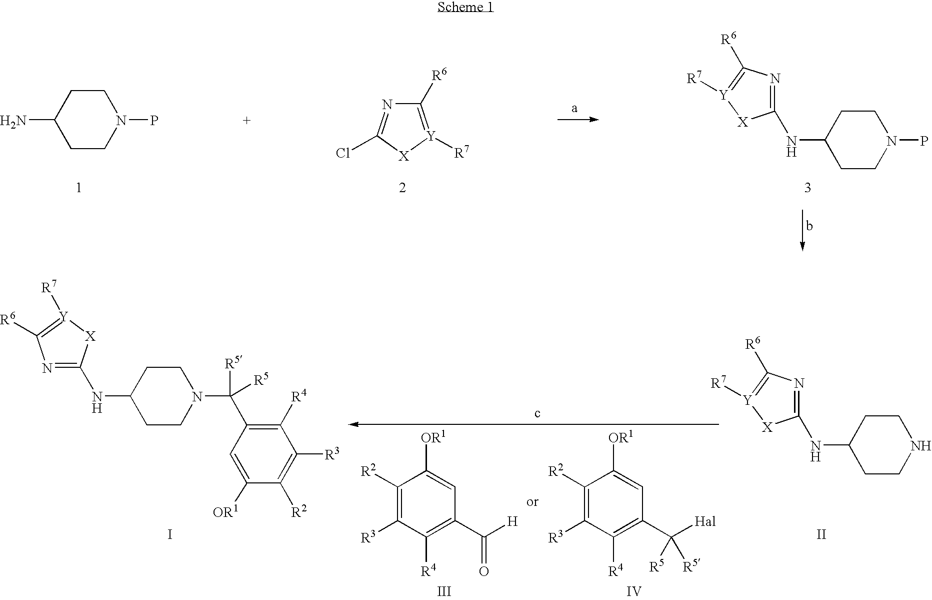

Alternatively, the reaction sequence can be performed in reverse order according to Scheme 2, namely first performing the alkylation (as previously described) with the secondary amino group of a suitably protected 4-amino piperidine (step a), e.g., relying on a tert-butyl carbamate protective group (see “Protective Groups in Organic Synthesis” above). Subsequent removal of the protecting group (step b) and coupling of the liberated amine V with the heteroaryl-chloride 2 as described before affords the desired compounds I (step c).

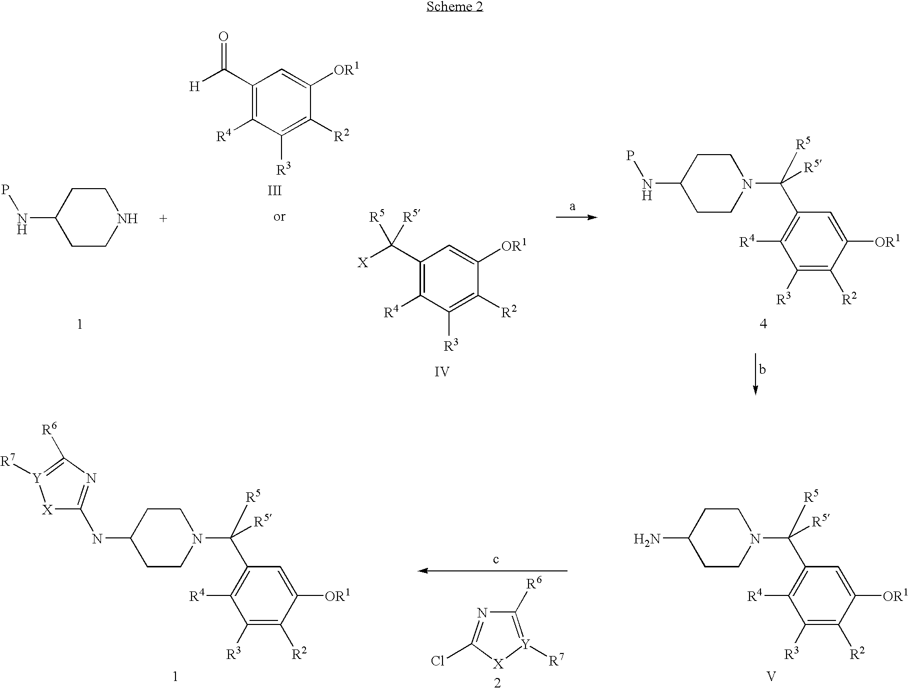

The benzimidazoles I (X═N, Y═C, R⁶ and R⁷ together are —CH═CH—CH═CH—) can be synthesized in close analogy by using a 2-chloro-benzimidazole as building block in Scheme 1 (compound 2) or Scheme 3 (compound 2). This intermediate is readily available by chlorination of the corresponding hydroxyl compound with POCl₃. Alkylation at nitrogen can optionally be performed before or thereafter. The same synthetic procedure is also applicable for the synthesis of aryloxadiazoles I (X═O, Y═N, R⁷—C=Aryl-C), cf. Tetrahedron Let. 1995, 36, 4471-4474).

The requisite aldehyde partners III are either commercially available or can be obtained by alkylation (Scheme 3, step a) of the phenolic aidehydes 8 with alkyl halides, alkyl mesylates or alkyl tosylates in a polar solvent, e.g., DMF or acetonitrile, and a suitable base, e.g., Cs₂CO₃, K₂CO₃, at room or elevated temperature, or by Mitsonobu reaction with alcohols activated by a mixture of triphenyiphosphine and diethyl- or di-tert-butyl-azodicarboxylate, or by analogous alkylation [or dialkylation] (step b) of the phenolic carboxylic esters [or acids] 5. In the latter case, reduction of the esters 6 by a suitable reducing agent, e.g., diisobutylaluminum hydride or LiAlH₄ at temperatures between −78° C. and ambient temperature in a solvent like THF will provide the alcohols 7 (step c). Those can then be oxidized to the aldehydes III, preferably with MnO₂ as oxidant in DCM (step d).

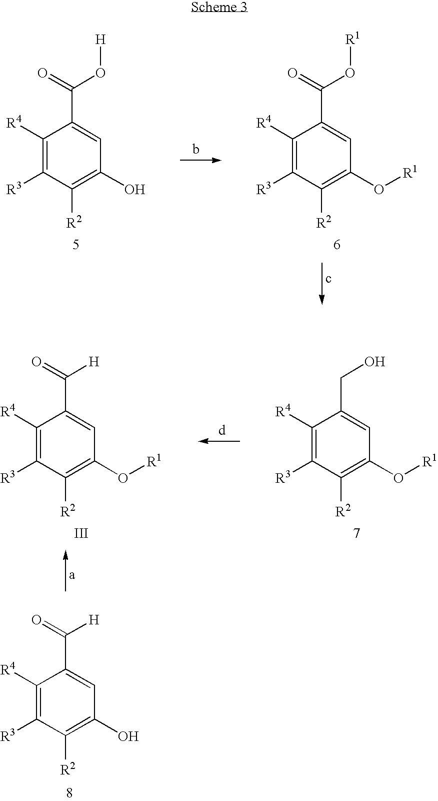

4-Halogen substituted acids 9 are known or can be prepared by methods well known in the art. Double alkylation, reduction and ensuing oxidation as described in Scheme 3 provides 4-halogen substituted aldehydes 12 (Scheme 4, steps a, b and c). 4-Fluoro-aldehydes 12 (Hal=F) react with imidazole or triazole in solvents like dimethylsulfoxide (DMSO) or dimethyltormamide (DMF) In the presence of a base like Cs₂CO₃, K₂CO₃, at room or elevated temperature to provide aldehydes III with R² representing a nitrogen linked imidazole or triazole moiety. 4-Iodo aldehydes 12 (Hal=I) react with cycloalkyl or aryl boronic acids In the presence of catalysts like (Ph₃P)₄Pd or Pd(OAc)₂/tricyclohexyl-phosphine and a base like K₃PO₄ in solvents or solvent mixtures including toluene, water, tetrahydrofuran, 1,2-dimethoxyethane or DMF to give aldehydes III with R² representing aryl or cycloalkyl (step d).

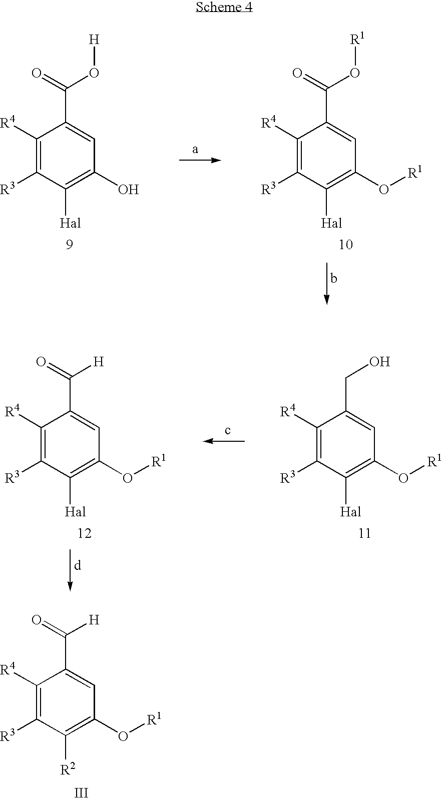

The requisite 4-aryl-oxazole chlorides 2 (R⁶=aryl, R⁷═H) can be synthesized according to Scheme 5. Optionally substituted phenacyl bromides 13, either commercially available or readily prepared by well known methods, can be transformed into the corresponding formates 14 by treatment with sodium formate in DMF following literature precedence (step a, J. Org. Chem. 1990, 55, 929-935). Heating with ammonium acetate in acetic acid delivers the key intermediates 15 (step b). The latter can then be deprotonated with a strong base, e g., n-BuLi or hexamethyldisilazide in THF at low temperature, and the resultant, labile anion quenched with a source of “Cl⁺”, e.g., hexachloroethane, to provide building block 2 (step c, Org. Lett. 2005, 7, 3351-3354).

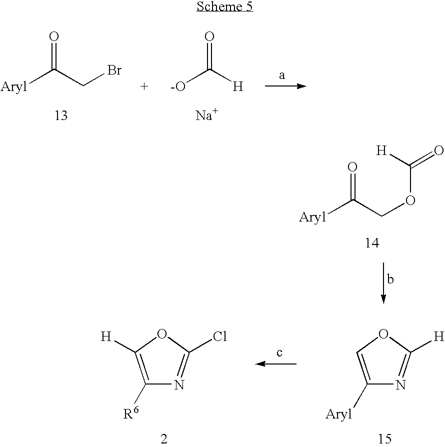

The requisite 5-aryl-oxazole chlorides 2 (R⁷=aryl, R⁶═H), on the other hand, can be prepared according to Scheme 6 relying on the van Leusen protocol (Tetrahedron Lett. 1972, 13, 2369-2372). Cyclocondensation of an appropriate benzaldehyde 16 with tosyl-methyl-isocyanide (TOSMIC) and a suitable base, e.g., K₂CO₃, in MeOH at reflux temperature delivers the 5-aryl-oxazoles 17, which are then chlorinated as described above to yield the necessary building blocks 2.

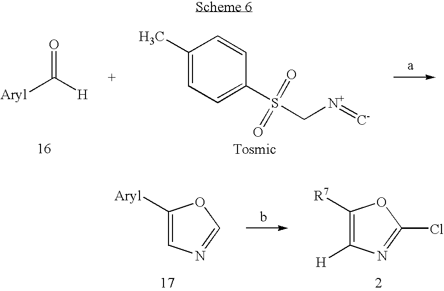

3-Aryl-[1,2,4]oxadiazoles containing a suitable leaving group can be prepared according to Scheme 7 by condensing an aryl-amidoxime 18 with e.g., trichloro-acetic acid methyl ester according to Durden and Heywood (J. Org. Chem. 1971, 36, 1306-1307).

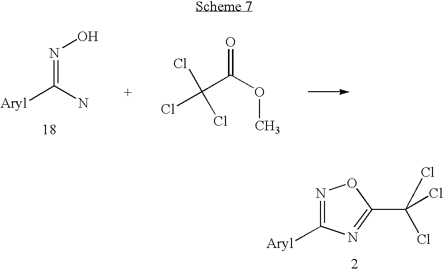

Additional synthetic procedures are described in more detail in the experimental part.

As described hereinbefore, it has been found that the compounds of formula I possess pharmaceutical activity, in particular they are modulators of somatostatin receptor activity. More particularly, the compounds of the present invention have been found to be antagonists of the somatostatin receptor subtype 5 (SSTR5).

The following tests were carried out in order to determine the activity of the compounds of formula I.

A CHO cell line stably transfected with a plasmid encoding the human subtype 5 somatostatin receptor (GenBank accession number D16827) was obtained from Euroscreen. Cells were cultured and used for binding and functional assays.

Membranes of these cells were prepared by sonication in the presence of protease inhibitors and subsequent fractionating centrifugation. The protein concentration in the membrane preparation was determined using a commercial kit (BCA kit, Pierce, USA). Membranes were stored at −80° C. until use. After thawing membranes were diluted in assay buffer (50 mM Tris-HCl at pH 7.4, 5 mM MgCl₂ and 0.20% BSA) and subjected to dounce homogenization.

For binding studies, 0.1 mL membrane suspension, corresponding to approximately 6×10⁻¹⁵ mol receptor, was incubated for 1 h at rt with 0.05 nM ¹²⁵I-labeled tracer (11-Tyr somatostatin-14, Perkin-Elmer) and either test compound in varying concentrations or, for the determination of non-specific binding, 0.001 mM non-labeled somatostatin-14. The incubation was stopped by filtration through GF/B glassfiber filters and washing with ice-cold wash buffer (50 mM Tris-HCl at pH 7.4). The bound radioactivity was measured after application of a scintillation cocktail (Microscint 40) and expressed as disintegrations per minute (dpm).

The receptor concentration was determined in a prior saturation experiment where a fixed, arbitrary amount of membranes was incubated with a concentration range of radio-labeled tracer. This allows estimating the total number of specific binding sites per amount of protein (i.e., Bₘₐₓ), typically between 1 and 5 pmol/mg.

The concentration of the test compound required to result in half maximal inhibition of binding of the radio-labeled tracer (IC₅₀) was estimated from a concentration-versus-dpm graph. The binding affinity (Kᵢ) was calculated from the IC₅₀ by applying the Cheng-Prussoff equation for single binding sites.

For functional experiments, 50,000 cells were incubated in Krebs Ringer HEPES buffer (115 mM NaCl, 4.7 mM KCl, 2.56 mM CaCl₂, 1.2 mM KH₂PO₄, 1.2 mM MgSO₄, 20 mM NaHCO₃ and 16 mM HEPES, adjusted to pH 7.4) supplemented with 1 mM IBMX and 0.1% BSA, then stimulated with 0.004 mM forskolin. Simultaneously with forskolin, test compound in varying concentrations was applied. Cells were then incubated for 20 minutes at 37° C. and 5% CO₂. Subsequently, cells were lysed and cAMP concentration measured using a fluorescence-based commercial kit according to the manufacturer (HitHunter cAMP, DiscoverX).

The concentration of the test compound to induce a half maximal effect (i.e., EC₅₀) as well as the efficacy as compared to 0.15 nM somatostatin-14 were determined from concentration-versus-fluorescence (arbitrary units) graphs. For the determination of potential antagonism, 0.15 nM somatostatin-14 was applied together with the test compound and the concentration of the test compounds to half maximally reverse the effect of somatostatin-14 (i.e., IC₅₀) was deduced from concentration-versus-fluorescence graphs.

The compounds of the present invention exhibit in a radioligand replacement assay Kᵢ values of 0.1 nM to 10 μM, preferably Kᵢ values of 0.1 nM to 500 nM and more preferably 0.1 nM to 100 nM for the human subtype 5 somatostatin receptor. The following table shows measured values for selected compounds of the present invention.

| | hSSTR5 |
|---|---|
| | Kᵢ (nmol/l) |
| Example 1 | 74 |
| Example 2 | 91 |
| Example 5 | 116 |
| Example 7 | 449 |
| Example 9 | 41 |
| Example 11 | 42 |
| Example 13 | 35 |
| Example 15 | 48 |
| Example 19 | 26 |
| Example 24 | 27 |
| Example 27 | 8 |
| Example 33 | 28 |
| Example 37 | 12 |
| Example 40 | 46 |
| Example 46 | 12 |
| Example 49 | 73 |

The compounds of formula I and their pharmaceutically acceptable salts and esters can be used as medicaments, e.g., in the form of pharmaceutical preparations for enteral, parenteral or topical administration. They can be administered, for example, perorally, e.g., in the form of tablets, coated tablets, dragdes, hard and soft gelatine capsules, solutions, emulsions or suspensions, rectally, e.g., in the form of suppositories, parenterally, e.g., in the form of injection solutions or infusion solutions, or topically, e.g., in the form of ointments, creams or oils.

The production of the pharmaceutical preparations can be effected in a manner which will be familiar to any person skilled in the art by bringing the described compounds of formula I and their pharmaceutically acceptable salts, into a galenical administration form together with suitable, non-toxic, inert, therapeutically compatible solid or liquid carrier materials and, if desired, usual pharmaceutical adjuvants.

Suitable carrier materials are not only inorganic, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers are, however, required in the case of soft gelatine capsules). Suitable carrier materials for the production of solutions and syrups are, for example, water, polyols, sucrose, invert sugar and the like. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are, for example, glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of the compounds of formula I can vary within wide limits depending on the disease to be controlled, the age and the individual condition of the patient and the mode of administration, and will, of course, be fitted to the individual requirements in each particular case. For adult patients a daily dosage of about 1 mg to about 1000 mg, especially about 1 mg to about 100 mg, comes into consideration. Depending on the dosage it is convenient to administer the daily dosage in several dosage units.

The pharmaceutical preparations conveniently contain about 0.1-500 mg, preferably 0.5-100 mg, of a compound of formula I.

The present invention will be further explained by reference to the following illustrative examples. They are, however, not intended to limit its scope in any manner.

### EXAMPLES

### Abbreviations

Ar=argon, BINAP=rac-2,2′-bis(diphenylphosphino)-1,1′-binaphthalene, Celite=filtration aid, DMF=N,N-dimethylformamide, DMSO=dimethyl sulfoxide, EI=electron impact (ionization), HPLC=high performance liquid chromatography, Hyflo Super Cel®=filtration aid (Fluka), ISP=ion spray positive (mode), NMR=nuclear magnetic resonance, MPLC=medium pressure liquid chromatography, MS=mass spectrum, P=protecting group, R=any group, rt=room temperature, THF=tetrahydrofuran, X=halogen.

The aldehyde intermediates were prepared following literature precedents or in analogy to literature precedents or as described below.

### 4-Chloro-3-ethoxy-benzaldehyde [CAS RN 85259-46-7]

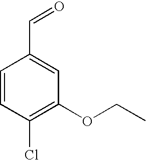

To a solution of 4-chloro-3-hydroxy-benzoic acid (3.0 g, 17.4 mmol, 1.0 equiv) in DMF (15 mL) was added K₂CO₃ (4.81 g, 34.8 mmol, 2.0 equiv) and ethyl iodide (4.03 mL, 5.97 g, 38.2 mmol, 2.2 equiv). The reaction mixture was stirred for 6 h at rt, diluted with water (20 mL) and extracted with ethyl acetate (3×50 mL). The organic phase was dried over Na₂SO₄ and concentrated to afford 3.6 g (91%) of 4chloro-3-ethoxy-benzoic acid ethyl ester. The crude ester was then dissolved in THF (20 mL) and cooled to −78° C. under Ar. A solution of diisobutylaluminum hydride (95 mL, 95.0 mmol, 6.0 equiv; 1.0 M solution in THF) was slowly added over a time period of 15 min, the cooling bath removed after completion of addition and the reaction allowed reaching 0° C. After stirring for 1 h, the reaction was cooled to −78° C. and the excess of hydride quenched by cautious addition of a solution of 1 M HCl (10 mL). The mixture was warmned up to rt, the organic phase separated and the aqueous layer extracted with ethyl acetate (3×100 mL). The combined organic phases were dried over Na₂SO₄ and concentrated by evaporation under reduced pressure providing 2.94 g (100%) of 4-chloro-3-ethoxy-benzyl alcohol. The crude alcohol (2.94 g, 15.75 mmol, 1.0 equiv) was dissolved in dichloromethane (15 mL) and activated MnO₂ (5.48 g, 63.0 mmol, 4.0 equiv) was added. The reaction mixture was stirred for 16 h, after which time the reaction was filtered through Hyflo Super Cel® and concentrated. The residue was purified by flash column chromatography on silica eluting with heptane/ethyl acetate (4:1) to yield 1.51 g (52%) of the title compound. ¹ H NMR (300 MHz, CDCl₃): δ 1.51 (t, J=7.1 Hz, 3H), 4.19 (q, J=7.1 Hz, 2H), 7.37-7.42 (m, 2H), 7.55 (d, J=9.0 Hz, 1H), 9.94 (s, 1H).

### 3-Isobutoxy-4-methoxy-benzaldehyde [CAS RN 57724-26-2]

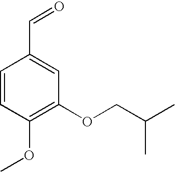

The title compound was prepared by reaction of isovanillin with 1-bromo-2-methyl propane as described in WO 04/000 806 A1 (Elbion AG).

### 3.5-Diisopropoxy-benzaldehyde [CAS RN 94169-64-9]

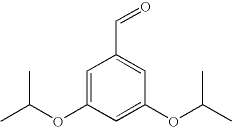

To a solution of 3,5-dihydroxy-benzaldehyde (5.0 g, 36.20 mmol, 1.0 equiv) in anhydrous DMF (30 mL) was added K₂CO₃ (15.0 g, 108.60 mmol, 3.0 equiv) and 2-bromo-propane (13.36 g, 10.20 mL, 108.60 mmol, 3.0 equiv) and the mixture stirred at 100° C. for 18 h. The K₂CO₃ was removed by filtration and the organic phase concentrated under reduced pressure. To the residue was added a sat. solution of NaCl (100 mL) and the solution extracted with ethyl acetate (3×100 mL). The combined organic phases were dried over MgSO₄ and the product purified by silica column chromatography using a MPLC system (CombiFlash Companion, Isco Inc.) eluting with a gradient of heptane/ethyl acetate affording 6.64 g (83%) of the title compound and 0.59 g (9%) of 3-hydroxy-5-isopropoxy-benzaldehyde. ¹H NMR (300 MHz, CDCl₃): δ 1.35 (d, J=6.1 Hz, 12H), 4.59 (hept, J=6.1 Hz, 2H), 6.66-6.68 (m, 1H), 6.96-6.97 (m, 2H), 9.88 (s, 1H). MS (ISP): 223.1 [M+H]⁺.

### 4-Chloro-3.5-diethoxy-benzaldehyde

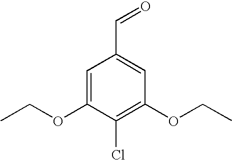

### Step 1: 4-Chloro-3,5-diethoxy-benzoic acid ethyl ester

To a solution of 4-amino-3,5-diethoxy-benzoic acid ethyl ester (5.1 g, 20.13 mmol, 1.0 equiv; prepared as described in I. Kompis and A. Wick Helv. Chim. Acta 1977, 60, 3025-3034) In water (40 mL) and 37% HCl (40 mL) at 0° C. was added sodium nitrite (1.67 g, 24.16 mmol, 1.2 equiv). After 10 min, copper(l) chloride (12.0 g, 120.81 mmol, 6.0 equiv) was added, the reaction mixture stirred tor an additional 5 h at 0° C. and then the ice bath removed. After stirring for 18 h, the crude reaction mixture was adjusted to pH=8 by addition of a solution of 1 M NaOH and the aqueous layer extraced with ethyl acetate (3×100 mL). The combined organic phases were dried over MgSO₄, concentrated by evaporation under reduced pressure and the crude material purified by silica column chromatography using a MPLC system (CombiFlash Companion, Isco Inc.) eluting with a gradient of heptane/ethyl acetate providing 5.0 g (91%) of the title compound. ¹H NMR (300 MHz, CDCl₃): δ 1.32 (t, J=7.0 Hz, 4H), 1.40 (t, J=7.0 Hz, 6H), 4.09 (q, J=7.0 Hz, 4H), 4.30 (q, J=7.18 (S, 2H). ¹³C NMR (75 MHz, CDCl₃): δ 13.33, 13.66, 60.29, 64.16, 105.75, 115.88, 128.25, 154.49, 165.01. MS (ISP): 273.3 [M+H]⁺.

### Step 2: (4-Chloro-3,5-diethoxy-phenyl)-methanol

To a solution of 4-chloro-3,5-diethoxy-benzoic acid ethyl ester (5.0 g, 18.33 mmol, 1.0 equiv) in dichloromethane (25 mL) was added slowly over a time period of 15 min under slight cooling to −30° C. a solution of diisobutylaluminum hydride (55.0 mL, 55.00 mmol, 3.0 equiv; 1.0 M solution in THF). After 30 min, the excess of hydride was quenched by cautious addition of methanol (10 mL) and water (2 mL). The mixture was stirred for 30 min, a solution of 1 M HCl was added and the aqueous layer extracted with ethyl acetate (3×100 mL). The combined organic phases were dried over MgSO₄ and concentrated by evaporation under reduced pressure providing 4.0 g (95%) of the title compound. ¹H NMR (300 MHz, CDCl₃): δ 1.45 (t, J=7.0 Hz, 6H), 1.93 (br s, 1H), 4.09 (q, J=7.0 Hz, 4H), 4.62 (s, 2H), 6.57 (s, 2H). ¹³C NMR (75 MHz, CDCl₃): δ 14.74, 64.96, 65.18, 104.30, 110.65, 140.29, 155.66. MS (ISP): 231.4 [M+H]⁺.

### Step 3: 4-Chloro-3,5-diethoxy-benzaldehyde

To a solution of (4-chloro-3,5-diethoxy-phenyl)-methanol (4.0 g, 17.34 mmol, 1.0 equiv) in THF (40 mL) was added activated MnO₂ (15.08 g, 173.4 mmol, 10.0 equiv) and the reaction mixture stirred for 18 h at rt. Filtration through Hyflo Super Cell® and purification of the crude material by silica column chromatography using a MPLC system (CombiFlash Companion, Isco Inc.) eluting with a gradient of heptane/ethyl acetate provided 3.7 g (92%) of the title compound. ¹H NMR (300 MHz, CDCl₃): δ 1.50 (t, J=7.0 Hz, 6H), 4.19 (q, J=7.0 Hz, 4H), 7.07 (s, 2H), 9.89 (s, 1H). ¹³C NMR (75 MHz, CDCl₃): δ 14.61, 65.22, 106.26, 118.64, 135.08, 156.22, 191.01. MS (EI): 229.4 [M]⁺.

### 2.6-Diethoxy-4′-fluoro-biphenyl-4-carbaldehyde

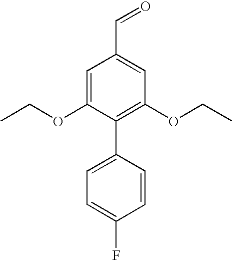

3,5-Diethoxy-4-iodo-benzaldehyde (14.05 g, 43.89 mmol, 1.0 equiv; prepared as described in WO 01/326 33 A1 (F. Hoffmann-La Roche AG); [CAS RN 338454-05-0]) was dissolved under Ar in toluene (180 mL) and water (20 mL) and treated successively with 4-fluorophenyl boronic acid (12.28 g, 87.78 mmol, 2.0 equiv), K₃PO₄ (50.12 g, 236.12 mmol, 5.38 equiv), tricyclohexylphosphine (2.80 g, 9.66 mmol, 0.22 equiv) and palladium(II) acetate (1.08 g, 4.83 mmol, 0.11 equiv). The reaction mixture was heated to 100° C. for 18 h under scrupulous exclusion of oxygen, when GC indicated the absence of starting iodo-compound. The reaction mixture was poured on crashed ice/NH₄Cl, extracted with ethyl acetate (2×200 mL) and the combined organic phases washed with a sat. solution of NaCl (2×100 mL) and water (2×100 mL). The organic phase was dried over Na₂SO₄, concentrated by evaporation under reduced pressure and the crude material purified by silica column chromatography eluting with a mixture of hexane/ethyl acetate (9:1). Recrystallization from hexane/ethyl acetate provided 10.44 g (83%) of the title compound as white crystals. MS (EI): 288.2 [M]⁺.

### 3.5-Diethoxy-4-fluoro-benzaldehyde

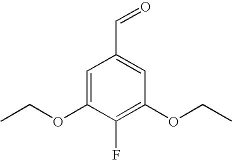

### Step 1: tert-Butyl-(4-fluoro-benzyloxy)-dimethyl-silane

To a solution of (4-fluoro-phenyl)-methanol (12.16 g, 96.4 mmol, 1.0 equiv) in anhydrous DMF (50 mL) at 0° C. under Ar was added imidazole (7.22 g, 106.1 mmol, 1.1 equiv) and tert-butyl-chloro-dimethyl-silane (15.99 g, 106.1 mmol, 1.1 equiv). After the addition was completed the cooling bath was removed and the reaction stirred for 18 h at rt. The reaction mixture was poured on ice, extracted with ethyl acetate (2×100 mL) and the combined organic phases washed with a sat. solution of Na₂CO₃ (2×100 mL) and a sat. solution of NaCl (2×100 mL). The organic phase was dried over Na₂SO₄, concentrated by evaporation under reduced pressure yielding a brown oil that was purified by high vacuum destination (bp 32-35° C. at 0.1 mbar) to give 23.0 g (99%) of the title compound. ¹H NMR (400 MHz, CDCl₃): δ0.00 (s, 6H), 0.84 (s, 9H), 4.60 (s, 2H), 6.89-6.94 (m, 2H), 7.16-7.20 (m, 2H). MS (EI): 183.1 [M-tert-Bu]⁺.

### Step 2: 5-(tert-Butyl-dimethyl-slianyloxymethyl)-2-fluoro-phenol

To a solution of tert-butyl-(4-fluoro-benzyloxy)-dimethyl-silane (5.00 g, 20.8 mmol, 1.0 equiv) in anhydrous THF (20 mL) was added at −78° C. under Ar a solution of sec-BuLi (17.6 mL, 22.8 mmol, 1.1 equiv; 1.3 M solution in hexane) within 30 min. Then a solution of trimethyl borate (2.37 mL, 2.20 g, 20.8 mmol, 1.0 equiv) in anhydrous THF (7.5 mL) was added slowly within 30 min and the cooling bath removed. A solution of conc. acetic acid (2.78 mL, 1.87 g, 31.2 mmol, 1.5 equiv) was slowly added followed by a solution of 35% hydrogen peroxide in water (2.0 mL, 2.23 g, 22.9 mmol, 1.1 equiv) and the reaction allowed to proceed at 0° C. for another 30 min. After stirring at rt for additional 4 h, the mixture was extracted with diethyl ether (2×100 mL) and the combined organic phases washed with a solution of 10% NaOH (2×100 mL) and a sat. solution of NaCG (2×100 mL). The organic phase was dried over Na₂SO₄, concentrated by evaporation under reduced pressure and the crude material purified with column chromatography on silica eluting with hexane/ethyl acetate (19:1) providing 4.80 g (90%) of the title compound. ¹H NMR (400 MHz, CDCl₃): δ 50.00 (s, 6H), 0.84 (s, 9H), 4.56 (s, 2H), 4.97 (br s, 1H), 6.68-6.72 (m, 1H), 6.87-6.94 (m, 2H). MS (EI): 256.2 [M]⁺.

### Step 3: 2-(tert-Butyl-dimethyl-silanyloxy)-4-(tert-butyl-dimethyl-silanyloxymethyl)-1-fluoro-benzene

To a solution of 5-(tert-butyl-dimethyl-silanyloxymethyl)-2-fluoro-phenol (4.60 g, 17.9 mmol, 1.0 equiv) in anhydrous DMF (20 mL) at 0° C. under Ar was added imidazole (1.34 g, 19.7 mmol, 1.1 equiv) and tert-butyl-chloro-dimethyl-silane (2.97 g, 19.7 mmol, 1.1 equiv). After the addition was completed the cooling bath was removed and the reaction stirred for 18 h at rt. The reaction mixture was poured on ice, extracted with ethyl acetate (2×100 mL) and the combined organic phases washed with a sat. solution of Na₂CO₃ (2×100 mL) and a sat. solution of NaCl (2×100 mL). The organic phase was dried over Na₂SO₄ and concentrated by evaporation under reduced pressure yielding 4.50 g (68%) of the title compound. ¹H NMR (400 MHz, CDCl₃): δ 50.00 (s, 6H), 0.10 (s, 6H), 0.85 (s, 9H), 0.92 (s, 9H), 4.55 (s, 2H), 6.71-6.74 (m, 1H), 6.80-6.83 (m, 1H), 6.87-6.92 (m, 1H). MS (EI): 370.2 [M]⁺.

### Step 4: 3-(tert-Butyl-dimethyl-silanyloxy)-5-(tert-butyl-dimethyl-silanyloxymethyl)-2-fluoro-phenol

To a solution of 2-(tert-butyl-dimethyl-silanyloxy)-4-(tert-butyl-dimethyl-silanyloxymethyl)-1-fluoro-benzene (23.70 g, 63.9 mmol, 1.0 equiv) in anhydrous THF (130 mL) was added at −78° C. under Ar a solution of sec-BuLi (54.5 mL, 71.6 mmol, 1.1 equiv; 1.3 M solution in hexane) within 30 min. Then a solution of trimethyl borate (7.13 mL, 6.64 g, 63.9 mmol, 1.0 equiv) in anhydrous THF (30 mL) was added slowly within 30 min and the cooling bath removed. A solution of conc. acetic acid (5.49 mL, 5.76 g, 95.9 mmol, 1.5 equiv) was slowly added followed by addition of a solution of 35% hydrogen peroxide in water (6.2 mL, 6.83 g, 70.3 mmol, 1.1 equiv) and the reaction allowed to proceed at 0° C. for another 30 min. After stirring at rt for additional 4 h, the mixture was extracted with diethyl ether (2×100 mL) and the combined organic phases washed with a solution of 10% NaOH (2×100 mL) and a sat. solution of NaCl (2×100 mL). The organic phase was dried over Na₂SO₄, concentrated by evaporation under reduced pressure, and the crude material was purified with column chromatography on silica eluting with hexane/ethyl acetate (19:1) providing 15.80 g (64%) of the title compound. ¹H NMR (400 MHz, CDCl₃): δ 0.00 (s, 6H), 0.10 (s, 6H), 0.85 (s, 9H), 0.91 (s, 9H), 4.50 (s, 2H), 4.93 (br s, 1H), 6.37 (d, J=5.6 Hz, 1H), 6.47 (d, J=5.6 Hz, 1H). MS (EI): 329.2 [M-tert-Bu]⁺.

### Step 5: tert-Butyl-(3,5-diethoxy-4-fluoro-benzyloxy)-dimethyl-silane

To a solution of 3-(tert-butyl-dimethyl-silanyloxy)-5-(tert-butyl-dimethyl-silanyloxymethyl)-2-fluoro-phenol (5.80 g, 15.0 mmol, 1.0 equiv) in DMF (60 mL) was added K₂CO₃ (4.56 g, 33.0 mmol, 2.2 equiv) and ethyl bromide (2.46 mL, 3.60 g, 33.0 mmol, 2.2 equiv) and the reaction mixture stirred under Ar at 60° C. for 5 h. The K₂CO₃ was removed by filtration, the crude reaction mixture concentrated by evaporation under reduced pressure, the residue extracted with ethyl acetate (3×100 mL), the combined organic phases washed with water (2×100 ml) and dried over Na₂SO₄. The solvent was removed by evaporation under reduced pressure and the crude material purified with column chromatography on silica eluting with hexane/ethyl acetate (99:1) providing 3.10 g (63%) of the title compound. ¹H NMR (400 MHz, CDCl₃): δ 0.00 (s, 6H), 0.85 (s, 9H), 1.33 (t, J=7.0 Hz, 6H), 4.00 (q, J=7.0 Hz, 4H), 4.55 (s, 2H), 6.47 (d, J=6.8 Hz, 2H). MS (ISP): 329.3 [M+H]⁺.

### Step 6: (3,5-Diethoxy-4-fluoro-phenyl)-methanol

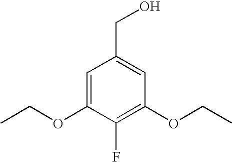

To a solution of tert-butyl-(3,5-diethoxy-4-fluoro-benzyloxy)-dimethyl-silane (1.20 g, 3.65 mmol, 1.0 equiv) in methanol (8 mL) was added Dowex 50W-X8 (0.33 g, cation exchange resin) and the reaction mixture stirred under Ar at rt for 22 h. The resin was removed by filtration and the reaction mixture concentrated by evaporation under reduced pressure yielding the title compound in quantitative yield (0.78 g). ¹H NMR (400 MHz, CDCl₃): δ 1.34 (t, J=7.0 Hz, 6H), 1.57 (t, J=5.4 Hz, 1H), 4.01 (q, J=7.0 Hz, 4H), 4.51 (d, J=5.4 Hz, 2H), 6.51 (d, J=6.8 Hz, 2H). MS (EI): 214.2 [M]⁺.

### Step 7: 3,5-Diethoxy-4-fluoro-benzaldehyde

To a solution of (3,5-diethoxy-4-fluoro-phenyl)-methanol (2.30 g, 10.7 mmol, 1.0 equiv) in 1,2-dichloroethane (50 mL) was added activated MnO₂ (2.89 g, 33.3 mmol, 3.1 equiv). The reaction mixture was stirred for 21 h at 50° C. and then filtered through Hyflo Super Cel® providing after evaporation of the solvent under reduced pressure 1.90 g (83%) of the title compound. ¹H NMR (400 MHz, CDCl₃): δ 1.38 (t, J=7.0 Hz, 6H), 4.09 (q, J=7.0 Hz, 4H), 7.04 (d, J=7.2 Hz, 2H), 9.75 (s, 1H). MS (EI): 212.1 [M]⁺.

### 4-Amino-3.5-diethoxy-benzaldehyde

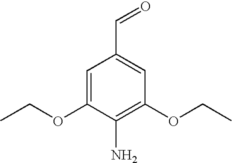

### Step 1: (4-Amino-3,5diethoxy-phenyl)-methanol

To a solution of 4-amino-3,5-diethoxy-benzoic acid ethyl ester (2.8 g, 11.05 mmol, 1.0 equiv; prepared as described in I. Kompis, A. Wick Helv. Chim. Acta 1977, 60, 3025-3034) in dichloromethane (50 mL) at 0° C. under Ar was slowly added diisobutylaluminum hydride (27.6 mL, 27.64 mmol, 2.5 equiv; 1.0 M solution in dichloromethane) over a time period of 15 min and the cooling bath removed on completion of addition. After stirring for 18 h, the excess of hydride was quenched by cautious addition of a sat. solution of potassium sodium tartrate (10 mL). The solidified mixture was extracted with dichloromethane (5×200 mL) and THF (2×150 mL), the combined organic phases washed with water (3×100 mL), dried over MgSO₄, concentrated by evaporation under reduced pressure and the crude material purified by column chromatography on silica eluting with a gradient of heptane/ethyl acetate (4:1→1:1) providing 1.10 g (47%) of the title compound. ¹H NMR (300 MHz, CDCl₃): δ 1.42 (t, J=7.0 Hz, 3H), 3.82 (br s, 2H), 4.05 (q, J=7.0 Hz, 2H), 4.54 (s, 2H), 6.50 (s, 2H). ¹³C NMR (75 MHz, CDCl₃): δ 15.03, 64.21, 66.00, 104.51, 125.44, 129.89, 146.71. MS (ISP): 211.9 [M+H]⁺.

### Step 2: 4-Amino-3,5-diethoxy-benzaldehyde

To a solution of (4-amino-3,5-diethoxy-phenyl)-methanol (0.79 g, 3.74 mmol, 1.0 equiv) in DMF (20 mL) was added activated MnO₂ (1.63 g, 18.70 mmol, 5.0 equiv). The reaction mixture was stirred for 24 h at rt, filtered through Hyflo Super Cel®, the filtrate was extracted with ethyl acetate (3×50 mL), and the combined organic phase was washed with water, dried over MgSO₄ and evaporated to dryness providing thereby 0.69 g (88%) of the title compound. ¹H NMR (300 MHz, DMSO): δ 1.46 (t, J=7.0 Hz, 3H), 4.15 (q, J=7.0 Hz, 2H), 4.50 (br s, 2H), 7.04 (s, 2H), 9.70 (s, 1H). MS (ISP): 210.0 [M+H]⁺.

### 3-Ethoxy-4-methyl-benzaldehyde [CAS RN 157143-20-9]

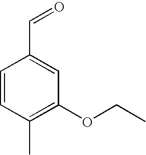

The title compound was prepared by reaction of commercially available 3-hydroxy-4-methyl-benzaldehyde with ethyl iodide in DMF using K₂CO₃ as base in analogy to the procedure described in M. J. Ashton, D. C. Cook, G. Fenton, J.-A. Karlsson, M. N. Palfreyman, D. Raebum, A. J. Ratcliffe, J. E. Souness, S. Thurairatnam and N. Vicker J. Med. Chem. 1994, 37, 1696-1703.

### 3-(2-Fluoro-ethoxy)-4-methoxy-benzaldehyde

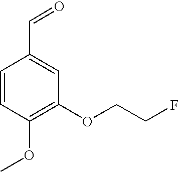

To a solution of 3-hydroxy-4-methoxy-benzaldehyde (10.0 g, 66.0 mmol, 1.0 equiv; commercially available) in anhydrous DMF (40 mL) was added K₂CO₃ (13.6 g, 99.0 mmol, 1.5 equiv) and 1-bromo-2-fluoro-ethane (9.2 mg, 72.0 mmol, 1.1 equiv) and the mixture stirred at rt for 48 h. The K₂CO₃ was removed by filtration and the organic phase concentrated under reduced pressure. To the residue was added a sat. solution of NaCl (100 mL) and the solution extracted with ethyl acetate (3×100 mL). The combined organic phases were dried over MgSO₄ and the product crystallized from a mixture of isopropanol/diethyl ether to yield 12.69 g (97%) of the title compound. ¹H NMR (300 MHz, DMSO): δ 3.89 (s, 3H), 4.24-4.27 (m, 1H), 4.34-4.37 (m, 1H), 4.67-4.70 (m, 1H), 4.83-4.86 (m, 1H), 7.20 (d, J=8.4 Hz, 1H), 7.43 (d, J=1.9 Hz, 1H), 7.59 (dd, J=8.4 Hz, J=1.9 Hz, 1H), 9.84 (s, 1H). MS (ISP): 198.6 [M+H]⁺.

### 3-Ethoxy-4-(1-ethyl-propoxy)-benzaldehyde

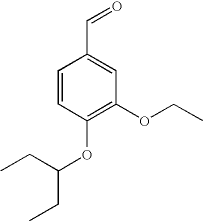

The title compound was prepared analogously to 3-ethoxy-4-methyl-benzaldehyde (see above) by reaction of 3-ethoxy-4-hydroxy-benzaldehyde with 3-bromo-pentane in DMF using K₂CO₃ as base.

MS (ISP): 237.1 [M+H]⁺.

### 3.5-Diethoxy-4-methyl-benzaldehyde

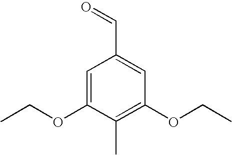

### Step 1: 3,5-Diethoxy-4-methyl-benzoic acid ethyl ester

3,5-Dihydroxy-4-methyl-benzoic acid (2.00 g, 11.9 mmol) was dissolved in 12 ml of DMF and treated successively with K₂CO₃ (6.58 g, 4 eq.) and ethyl iodide (3.84 ml, 4 eq.), and the reaction allowed to proceed over night at 45-50° C. Cooling, pouring onto crashed ice/NH₄Cl, twofold extraction with AcOEt, washing with water, drying over sodium sulfate, and evaporation of the solvents, followed by short flash chromatography (SiO₂, hexane/AcOEt=9/1), gave 2.099 g of the title compound as white solid.

MS (ISP): 253.1 [M+H]⁺.

### Step 2: (3,5-Diethoxy-4-methyl-phenyl)methanol

To the above synthesized 3,5-diethoxy-4-methyl-benzoic acid ethyl ester (2.090 g, 8.28 mmol), dissolved in 40 ml of abs. THF, was added drop wise at −10° C. DIBAL-H solution in hexane (29.0 ml 1M, 3.5 eq.). After additional 80 Min. stirring at RT, the reaction mixture was carefully poured onto crashed ice/dil HCl-solution, twofold extracted with AcOEt, washed with water, dried over sodium sulfate, and evaporated to dryness to afford finally 1.77 g of the title compound as white solid.

MS (EI): 210.2 [M]⁺.

### Step 3: 3,5-Diethoxy-4-methyl-benzaldehyde

The above prepared (3,5-diethoxy-4-methyl-phenyl)-methanol_(1.77 g, 8.28 mmol) was dissolved in 80 ml of dichloromethane and treated with MnO₂ (14.4 g, 20 eq.). After vigorous stirring over night at ambient temperature, the reaction mixture was filtered over a pad of Celite, rinsed generously with dichloromethane, and evaporated to dryness. Ensuing flash chromatography (SiO₂, hexane/AcOEt=9/1), yielded then 1.454 g of the title compound as light yellow solid.

MS (EI): 208.2 [M]⁺.

### 8-Ethoxy-2.2-dimethyl-2H-chromene-6-carbaldehyde [CAS RN 210404-30-9]

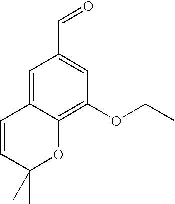

The title compound was prepared according to WO 01/083 476 A1 (Hoffmann-La Roche AG).

### Example 1

### [1-(3-Ethoxy-4-methyl-benzyl)-piperidin-4-yl]-(3-phenyl-[1,2,4]oxadiazol-5-yl)-amine

### Step 1: 3-Phenyl-5-trichloromethyl-[1,2,4]oxadiazole [CAS RN 1208-05-5]

The title compound was prepared as described in J. A. Durden Jr. and D. L. Heywood J. Org. Chem. 1965, 30, 4359-4361.

### Step 2: 4-(3-Phenyl-[1,2,4]oxadiazol-5-ylamino)-piperidine-1-carboxylic acid tert-butyl ester

A solution of 3-phenyl-5-trichloromethyl-[1,2,4]oxadiazole (1.00 g, 3.80 mmol, 1.0 equiv), 4-amino-piperidine-1-carboxylic acid tert-butyl ester (0.84 g, 4.17 mmol, 1.1 equiv) and NaHCO₃ (0.64 g, 7.60 mmol, 2.0 equiv) in N-methylpyrrolidone (10 mL) was heated by microwave irradiation to 120° C. for 20 min. The solvent was removed under reduced pressure and the crude reaction product extracted from a sat. solution of NaCl (50 mL) with diethyl ether (3×50 mL). The combined organic phases were dried over MgSO₄ and concentrated by evaporation under reduced pressure. The crude material was purified by silica column chromatography using a MPLC system (CombiFlash Companion, Isco Inc.) eluting with a gradient of heptane/ethyl acetate to give 0.64 g (49%) of the title compound.

MS (ISP): 345.1 [M+H]⁺.

### Step 3: (3-Phenyl-[1,2,4]oxadiazol-5-yl)-piperidin-4-yl-amine diyhdrochloride

A solution of 4-(3-phenyl-[1,2,4]oxadiazol-5-ylamino)-piperidine-1-carboxylic acid tert-butyl ester (0.64 g, 1.86 mmol) in 4 M HCl in dioxane (20 mL) was stirred at rt for 2 h. The solvent was removed under reduced pressure and the crude product used in the consecutive step without further purification assuming quantitative deprotection and formation of the dihydrochloride salt.

MS (ISP): 245.1 [M+H]⁺.
Step 4: [1-(3-Ethoxy-4-methyl-benzyl)-piperidin-4-yl]-(3-phenyl-[1,2,4]oxadiazol-5-yl)-amine

To a solution of (3-phenyl-[1,2,4]oxadiazol-5-yl)-piperidin-4-yl-amine diyhdrochloride (54.6 mg, 0.18 mmol, 1.0 equiv) in ethanol (1 mL), acetic acid (72.1 mg, 1.2 mmol, 8.0 equiv) and N-ethyidiisopropylamine (77.6 mg, 0.6 mmol, 4.0 equiv) was added 3-ethoxy-4-methyl-benzaldehyde (35.5 mg, 0.22 mmol, 1.2 equiv) and the mixture stirred at 55° C. After 1 h, sodium cyanoborohydride (47.1 mg, 0.75 mmol, 5.0 equiv), dissolved in ethanol (0.5 mL), was added and the mixture stirred at 55° C. over night. Removal of the solvent under reduced pressure and purification by preparative HPLC on reversed phase eluting with a gradient of acetonitriletwater provided 26.8 mg (38%) of the title compound.

MS (ISP): 393.3 [M+H]⁺.

### Example 2

### [1-(4-Chloro-3-ethoxy-benzyl)-piperidin-4-yl]-(3-phenyl-[1,2,4]oxadiazol-5-yl)-amine

The title compound was prepared in analogy to Example 1, but using in the reductive amination step 4-chloro-3ethoxy-benzaldehyde instead of 3-ethoxy-4-methyl-benzaldehyde.

MS (ISP): 413.3 [M+H]⁺.

### Example 3

### 2-Ethoxy-4-[4-(3-phenyl-[1,2,4]oxadiazol-5-ylamino)-piperidin-1-ylmethyl]-phenol

The title compound was prepared in analogy to Example 1, but using in the reductive amination step 3-ethoxy-4-hydroxy-benzaldehyde (commercially available) instead of 3-ethoxy-4-methyl-benzaldehyde.

MS (ISP): 395.3 [M+H]⁺.

### Example 4

### [1-(3-Ethoxy-4-methoxy-benzyl)-piperidin-4-yl]-(3-phenyl-[1,2,4]oxadiazol-5-yl)-amine

The title compound was prepared in analogy to Example 1, but using in the reductive amination step 3-ethoxy-4-methoxy-benzaldehyde (commercially available) instead of 3-ethoxy-4-methyl-benzaldehyde.

MS (ISP): 409.4 [M+H]⁺.

### Example 5

### [1-(3-Isobutoxy-4-methoxy-benzyl)piperidin-4-yl]-(3-phenyl-[1,2,4]oxadiazol-5-yl)-amine

The title compound was prepared in analogy to Example 1, but using in the reductive amination step 3-isobutoxy-4-methoxy-benzaldehyde instead of 3-ethoxy-4-methyl-benzaldehyde.

MS (ISP): 437.4 [M+H]⁺.

### Example 6

### {1-[3-(2-Fluoro-ethoxy)-4-methoxy-benzyl]-piperidin-4-yl}-(3-phenyl-[1,2,4]oxadiazol-5-yl)-amine

The title compound was prepared in analogy to Example 1, but using in the reductive amination step 3-(2-fluoro-ethoxy)-4-methoxy-benzaldehyde instead of 3-ethoxy-4-methyl-benzaldehyde.

MS (ISP): 427.2 [M+H]⁺.

### Example 7

### [1-(8-Ethoxy-2,2-dimethyl-2H-chromen-6-ylmethyl)-piperidin-4-yl]-(3-phenyl-[1,2,4]oxadiazol-5-yl)-amine

The title compound was prepared in analogy to Example 1, but using in the reductive amination step 8-ethoxy-2,2-dimethyl-2H-chromene-6-carbaldehyde instead of 3-ethoxy-4-methyl-benzaldehyde.

MS (ISP): 461.4 [M+H]⁺.

### Example 8

### [1-(3,5-Diethoxy-4-fluoro-benzyl)-piperidin-4-yl]-(3-phenyl-[1,2,4]oxadiazol-5-yl)-amine

The title compound was prepared in analogy to Example 1,but using in the reductive amination step 3,5diethoxy-4-fluoro-benzaldehyde instead of 3-ethoxy-4-methyl-benzaldehyde.

MS (ISP): 441.4 [M+H]⁺.

### Example 9

### [1-(4-Amino-3,5-diethoxy-benzyl)-piperidin-4-yl]-(3-phenyl-[1,2,4]oxadiazol-5-yl)-amine

The title compound was prepared In analogy to Example 1, but using in the reductive amination step 4-amino-3,5-diethoxy-benzaldehyde instead of 3-ethoxy-4-methyl-benzaldehyde.

MS (ISP): 438.4 [M+H]⁺.

### Example 10

### (1H-Benzoimidazol-2-yl)-{1-[3ethoxy-4-(1-ethyl-propoxy)-benzyl]-piperidin-4-yl}-amine

The compound was prepared in analogy to Example 1, but using in the reductive amination step 3-ethoxy-4-(1-ethyl-propoxy)-benzaldehyde instead of 3-ethoxy-4-methyl-benzaldehyde.

MS (ISP): 437.4 [M+H]⁺.

The former, necessary intermediate

### (1H-Benzoimidazol-2-yl)-piperidin-4-yl-amine dihydrochloride

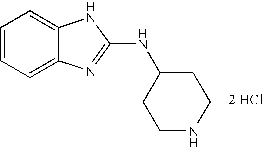

was synthesized as described in GB 237 3186 A (AstraZeneca AB).

### Example 11

### [1-(4-Chloro-3-ethoxy-benzyl)-piperidin-4-yl]-(1-methyl-1H-benzoimidazol-2-yl)-amine

### Step 1: 2-Chloro-1-methyl-1H-benzoimidazole [CAS RN 1849-02-1]

The title compound was prepared as described in D. Harrison, J. T. Ralph and A. C. B. Smith J. Chem. Soc. 1963, 2930-2937.

### Step 2: 4-(1-Methyl-1H-benzoimidazol-2-ylamino)-piperidine-1-carboxylic acid tert-butyl ester

Chloro-1-methyl-1H-benzimidazole (3.7 g, 22.2 mmol, 1.0 equiv), tris(dibenzylidene acetone)dipalladium(0) (4.9 g. 5.4 mmol, 0.24 equiv), BINAP (10.1 g, 16.2 mmol, 0.73 equiv) and sodium tert-butoxide (2.9 g, 31 mmol, 1.40 equiv) were charged into a round-bottomed flask. Anhydrous toluene (78 mL) and 4-amino-piperidine-1-carboxylic acid tert-butyl ester (5.3 g, 26.6 mmol, 1.20 equiv) were added to the flask and N₂ was bubbled through the reaction mixture for 30 min. The reaction mixture was stirred at 85° C. for 16 h. The suspension was filtered through Celite and extracted with 1 M aqueous HCl (3×20 mL). The combined aqueous layers were washed with dichloromethane (20 mL) then adjusted to pH 7 with 1 M NaOH solution. The aqueous layer was extracted with dichloromethane (3×20 mL), the combined organic extracts dried over Na₂SO₄ and evaporated under reduced pressure to give an amber oil. The oil was purified by silica column chromatography eluting with a mixture of heptane/ethyl acetate/acetic acid (50:49:1) to obtain 1.53 g (21%) of the title compound.

MS (ISP): 331.4 [M+H]⁺.

### Step 3: (1-Methyl-1H-benzoimidazol-2-yl)-piperidin-4-yl-amine dihydrochloride

A solution of 4-(1-methyl-1H-benzoimidazol-2-ylamino)-piperidine-1-carboxylic acid tert-butyl ester (1.53 g, 4.63 mmol) in 4 M HCl in dioxane (20 mL) was stirred at rt for 2 h. The solvent was removed under reduced pressure and the crude product used in the consecutive step without further purification assuming quantitative deprotection and formation of the dihydrochloride salt.

MS (ISP): 231.3 [M+H]⁺.

### Step 4: [1-(4-Chloro-3-ethoxy-benzyl)-piperidin-4-yl]-(1-methyl-1H-benzoimidazol-2-yl)-amine

The title compound was prepared in analogy to Example 1, but using in the reductive amination step (1-methyl-1H-benzoimidazol-2-yl)-piperidin4-yl-amine dihydrochloride instead of (3-phenyl-[1,2,4]oxadiazol-5-yl)-piperidin-4-yl-amine diyhdrochloride and 4-chloro-3-ethoxy-benzaldehyde instead of 3-ethoxy-4-methyl-benzaldehyde.

MS (ISP): 399.2 [M+H]⁺.

### Example 12

### [1-(3-Ethoxy-4-methoxy-benzyl)-piperidin-4-yl]-(1-methyl-1H-benzoimidazol-2-yl)-amine

The title compound was prepared in analogy to Example 11, but using in the reductive amination step 3-ethoxy-4-methoxy-benzaldehyde (commercially available) instead of 4-chloro-3-ethoxy-benzaldehyde.

MS (ISP): 395.2 [M+H]⁺.

### Example 13

### [1-(3,5-Diisopropoxy-benzyl)-piperidin-4-yl]-(1-methyl-1H-benzoimidazol-2-yl)-amine

The title compound was prepared in analogy to Example 11, but using in the reductive amination step 3,5-diisopropoxy-benzaldehyde instead of 4-chloro-3-ethoxy-benzaldehyde.

MS (ISP): 437.3 [M+H]⁺.

### Example 14

### [1-(3,5-Diethoxy-4-fluoro-benzyl)-piperidin-4-yl]-(1-methyl-1H-benzoimidazol-2-yl)-amine

The title compound was prepared in analogy to Example 11, but using in the reductive amination step 3,5-diethoxy-4-fluoro-benzaldehyde instead of 4-chloro-3-ethoxy-benzaldehyde.

MS (ISP): 427.3 [M+H]⁺.

### Example 15

### [1-(2,6-Diethoxy-4′-fluoro-biphenyl-4-ylmethyl)-piperidin-4-yl]-[4-(3-methoxy-phenyl)-oxazol-2-yl]-amine

### Step 1: Formic acid 3-(3-methoxy-phenyl)-3-oxo-propyl ester

2-Bromo-1-(3-methoxy-phenyl)-ethanone (4.17 g, 18 mmol) was dissolved in abs. DMF and treated over night at ambient temperature with sodium formiate (1.609 g, 1.3 eq.). Pouring onto crashed ice, twofold extraction with ethyl acetate, washing with water, drying over sodium sulfate, and evaporation of the solvents left 3.64 g of the title product as light brown oil.

MS (ISP): 195.1 [M+H]⁺.

### Step 2: 4-(3-Methoxy-phenyl)-oxazole

The above prepared formic acid 3-(3-methoxy-phenyl)-3-oxo-propyl ester (3.64 g, 18 mmol) was dissolved in 36 ml of acetic acid, treated with ammonium acetate (7.02 g, 5 eq.), and kept for 1.5 h at 115° C. Pouring onto crashed ice, twofold extraction with AcOEt, washing with water, drying over sodium sulfate, and evaporation of the solvents, followed by flash chromatography (SiO₂, hexane/AcOEt=85/15), yielded 0.606 g of the title compound as red oil.

MS (ISP): 176.4 [M+H]⁺.

### Step 3: 2-Chloro-4-(3-methoxy-phenyl)-oxazole

The above prepared 4-(3-methoxy-phenyl)-oxazole (0.606 g, 3.46 mmol) was dissolved in 8.5 ml of abs. THF, cooled to −78° C., and deprotonated by adding via syringe 2.54 ml of nBuLi (1.5 M (hexane), 1.1 eq.). Hexachloroethane (1.228 g, 1.5 eq.) was then added all at once as solid and the mixture kept for 0.5 h at −78° C. before allowing to reach rt. Pouring onto crashed ice, twofold extraction with AcOEt, washing with water and brine, drying over sodium sulfate, and evaporation of the solvents, followed by flash chromatography (SiO₂, hexane/AcOEt=9/1), produced 0.605 g of the title compound as red oil.

MS (ISP): 210.1 [M+H]⁺.

### Step 4: 4-[4-(3-Methoxy-phenyl)-oxazol-2-ylamino]-piperidine-1-carboxylic acid tert-butyl ester

The above prepared 2-chloro-4-(3-methoxy-phenyl)-oxazole (0.200 g, 0.95 mmol) was condensed with 4-amino-piperidine-1-carboxylic acid tert-butyl ester (0.287 g, 1.5 eq.) in the presence of 2 eq. of N-ethyidiisopropylamine (0.33 ml) under microwave irradiation in 3 ml of acetonitrile at 190° C. for 2.5 h. Cooling, pouring onto crashed ice, twofold extraction with AcOEt, washing with brine, drying over sodium sulfate, and evaporation of the solvents, followed by silica column chromatography (hexane/ethyl acetate=1:1), yielded 0.200 g of the title compound as yellow oil.

MS (ISP): 374.4 [M+H]⁺.

### Step 5: [4-(3-Methoxy-phenyl)-oxazol-2-yl]-piperidin-4-yl-amine dihydrochloride

The above prepared 4-[4-(3-methoxy-phenyl)-oxazol-2-ylamino]-piperidine-1-carboxylic acid tert-butyl ester (0.200 g, 0.536 mmol) was treated with 4 M HCl in dioxane (2.7 mL). After stirring for 2 h at ambient temperature, TLC indicated the absence of starting material.

Evaporation of all volatiles left 0.202 g of the title compound as dihydrochloride as off-white solid.

MS (ISP): 274.4 [M+H]⁺.

### Step 6: [1-(2,6-Diethoxy-4′-fluoro-biphenyl-4-ylmethyl)-piperidin-4-yl]-[4-(3-methoxy-phenyl)-oxazol-2-yl]-amine

The above prepared [4-(3-methoxy-phenyl)-oxazol-2-yl]-piperidin-4-yl-amine (0.101 g, 0.27 mmol, corrected) was dissolved in 3.5 ml of iPrOH and treated successively with 2,6-diethoxy-4′-fluoro-biphenyl-4-carbaldehyde (0.077 g, 1.0 equiv.), titanium tetra-isopropoxide (0.39 ml, 5 eq.), and N-ethyl diisopropylamine (0.14 ml, 3 eq.), followed by NaCNBH₃ (0.034 g, 2 eq.) after stirring for 2 h. The reaction mixture was allowed to react over the weekend and then poured directly onto a flash column (SiO2). Elution with AcOEt, followed by a second column with the isolated crude product (SiO₂, AcOEt/5% MeOH) delivered finally 0.050 g of the title compound as yellow foam.

MS (ISP): 546.4 [M+H]⁺.

### Example 16

### [1-(3,5-Diethoxy-4-fluoro-benzyl)-piperdin-4-yl]-[4-(3-methoxy-phenyl)-oxazol-2-yl]-amine

The title compound was prepared in analogy to Example 15, but using in the reductive amination step 3,5-diethoxy-4-fluoro-benzaldehyde instead of 2,6-diethoxy-4′-fluoro-biphenyl-4-carbaldehyde, as yellow oil.

MS (ISP): 470.1 [M+H]⁺.

### Example 17

### [1-(2,6-Diethoxy-4′-fluoro-biphenyl-4-ylmethyl)-piperidin-4-yl]-[4-(4-(fluoro-phenyl)-oxazol-2-yl]-amine

The title compound was prepared in analogy to Example 15, but using as building block in step 4 2-chloro-4-(4-fluoro-phenyl)-oxazole instead of 4-(3-methoxy-phenyl)-oxazole, as white foam.

MS (ISP): 534.3 [M+H]⁺.

The former, necessary intermediate

### 2-Chloro-4-(4-fluoro-phenyl)-oxazole

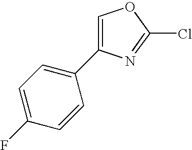

was synthesized as described in Example 15, step 1-3, but starting the reaction sequence with 2-bromo-1-(4-fluoro-phenyl)-ethanone instead of 2-bromo-1-(3-methoxy-phenyl)-ethanone, as off-white crystals.

MS (EI): 197.1 [M]⁺.

### Example 18

### [1-(2-Ethoxy-4′-fluoro-biphenyl-4-ylmethyl)-piperidin-4-yl]-[4-(4-fluoro-phenyl)-oxazol-2-yl]-amine

The title compound was prepared in analogy to Example 17, but using in the reductive amination step 2-ethoxy-4′-fluoro-biphenyl-4-carbaldehyde instead of 2,6-diethoxy-4′-fluoro-biphenyl-4-carbaldehyde, as colorless gum.

MS (ISP): 490.3 [M+H]^{+.}

### Example 19

### [1-(3,5-Diethoxy-fluoro-benzyl)-piperidin-4yl]-[4-(4-fluoro-phenyl)-oxazol-2-yl]-amine

The title compound was prepared in analogy to Example 18, but using in the reductive amination step 3,5-diethoxy-4-fluoro-benzaldehyde instead of 2,6-diethoxy-4′-fluoro-biphenyl-4-carbaldehyde, as colorless gum.

MS (ISP): 458.2 [M+H]⁺.

### Example 20

### [1-(4-Chloro-3,5-diethoxy-benzyl)-piperidin-4-yl]-[4-(3-methoxy-phenyl)-oxazol-2-yl]-amine

The title compound was prepared in analogy to Example 15, but using in the reductive amination step 4-chloro-3,5-diethoxy-benzaidehyde instead of 2,6-diethoxy-4′-fluoro-biphenyl-4-carbaldehyde, as white foam.

MS (ISP): 486.2 [M+H]⁺.

### Example 21

### [1-(3-Ethoxy-4-methyl-benzyl)-piperidin-4-yl]-[4-(3-methoxy-phenyl)-oxazol-2-yl]-amine

The title compound was prepared in analogy to Example 15, but using in the reductive amination step 3-ethoxy-4-methyl-benzaldehyde instead of 2,6-diethoxy-4′-fluoro-biphenyl-4-carbaldehyde, as light yellow oil.

MS (ISP): 422.12 [M+H]⁺.

### Example 22

### [1-(3,5-Diisopropoxy-benzyl)-piperidin-4-yl]-[4-(4-fluoro-phenyl-oxazol-2-yl]-amine

The title compound was prepared in analogy to Example 17, but using in the reductive amination step 3,5-diisopropoxy-benzaldehyde instead of 3,5-diethoxy-4-fluoro-benzaldehyde, as light yellow gum.

MS (ISP): 468.2 [M+H]⁺.

### Example 23

### [1-(4-Chloro-3,5-diethoxy-benzyl)-piperidin-4-yl]-[4-(4-fluoro-phenyl)-oxazol-2-yl]-amine

The title compound was prepared in analogy to Example 17, but using in the reductive amination step 4-chloro-3,5-diethoxy-benzaldehyde instead of 3,5-diisopropoxy-benzaldehyde, as white foam.

MS (ISP): 474.0 [M+H]⁺.

### Example 24

### [1-(2,6-Diethoxy-4′-fluoro-biphenyl-4-ylmethyl)-piperidin-4-yl]-[(4-phenyl-oxazol-2-yl)-amine

The compound was prepared in analogy to Example 15, but using as building block in step 4 2-chloro-4-phenyl-oxazole instead of 2-chloro-4-(3-methoxy-phenyl)-oxazole, as light yellow gum.

MS (ISP): 516.3 [M+H]⁺.

The former, necessary intermediate

### 2-Chloro-4-phenyl-oxazole

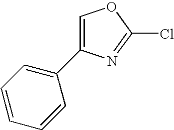

was synthesized as described in Example 15, step 1-3, but starting the reaction sequence with 2-bromo-1-phenyl-ethanone instead of 2-bromo-1-(3-methoxy-phenyl)-ethanone, as off-white crystals.

MS (EI): 179.1 [M]⁺.

### Example 25

### [1-(4-Chloro-3,5-diethoxy-benzyl)-piperidin-4-yl]-(4-phenyl-oxazol-2-yl)-amine

The title compound was prepared in analogy to Example 24, but using in the reductive amination step 4-chloro-3,5-diethoxy-benzaldehyde instead of 2,6-diethoxy-4′-fluoro-biphenyl-4-carbaldehyde, as light yellow gum.

MS (ISP): 456.3 [M+H]⁺.

### Example 26

### [1-(3,5-Diisopropoxy-enzyl)-piperidin-4-yl]-(4-phenyl-oxazol-2-yl)-amine

The title compound was prepared in analogy to Example 24, but using in the reductive amination step 3,5-diisopropoxy-benzaldehyde instead of 2,6-diethoxy-4′-fluoro-biphenyl-4-carbaldehyde, as colorless gum.

MS (ISP): 450.1 [M+H]⁺.

### Example 27

### [1-(4-Chloro-3,5-diethoxy-benzyl)-piperidin-4-yl]-[4(4-chloro-phenyl)oxazol-2-yl]-amine

The compound was prepared in analogy to Example 15, but using as building block in step 4 2-chloro-4-(4-chloro-phenyl)-oxazole instead of 2-chloro-4-(3-methoxy-phenyl)-oxazole, as light yellow gum.

MS (ISP): 490.2 [M+H]⁺.

The former, necessary intermediate

### 2-Chloro-4-(4-chloro-phenyl)-oxazole

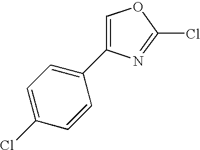

was synthesized as described in example 15, step 1-3, but starting the reaction sequence with 2-bromo-1-(4-chloro-phenyl)-ethanone instead of 2-bromo-1-(3-methoxy-phenyl)-ethanone, as off-white crystals.

MS (EI): 213.1 [M]⁺.

### Example 28

### [4-(4-chloro-phenyl)-oxazol-2-yl]-[1(2.6-diethoxy-4′-fluoro-biphenyl-4-ylmethyl)-piperidin-4-yl]-amine

The compound was prepared in analogy to Example 27, but using in the reductive amination step 2,6-diethoxy-4′-fluoro-biphenyl-4-carbaldehyde instead of 4-chloro-3,5-diethoxy-benzaldehyde, as off-white foam.

MS (ISP): 450.2 [M+H]⁺.

### Example 29

### [4-(4-Chloro-phenyl)-oxazol-2-yl]-[1-(3,5-diethoxy-4-fluoro-benzyl)-piperidin-4-yl]-amine

The title compound was prepared in analogy to Example 27, but using in the reductive amination step 3,5-diethoxy-4-fluoro-benzaldehyde instead of 4-chloro-3,5-diethoxy-bonzaldehyde, as yellow oil.

MS (ISP): 474.0 [M+H]⁺.

### Example 30

### [1-(3,5-Diethoxy-4-fluoro-benzyl)-piperidin-4-yl]-(4-phenyl-oxazol-2-yl)-amine

The compound was prepared in analogy to Example 24, but using in the reductive amination step 3,5-diethoxy-4-fluoro-benzaldehyde instead of 2,6-diethoxy-4′-fluoro-biphenyl-4-carbaldehyde, as light yellow solid.

MS (ISP): 440.3 [M+H]⁺.

### Example 31

### [1-(2-Ethoxy-4′-fluoro-biphenyl4-ylmethyl)-piperidin-4-yl]-[5-(4-fluoro-phenyl)-oxazol-2-yl]-amine

### Step 1: 5-(4-Fluoro-phenyl)-oxazole

To 4-fluorobenzaldehyde (1.50 g, 12 mmol) and tosylmethyl isocyanide (2.384, 1 eq.) in 15 mL of MeOH was added K₂CO₃ (3.34 g, 2 eq.), and the mixture was heated to gentle reflux for 2 h. Cooling, pouring onto crashed ice/NH₄Cl, twofold extraction with AcOEt, washing with water and brine, drying over magnesium sulfate, and evaporation of the solvents, followed by flash chromatography (SiO₂, hexane/AcOEt=85/15), yielded 1.83 g of the title compound as light yellow crystals.

MS (EI): 163.1 [M]⁺.

### Step 2: 2-Chloro-5-(4-fluoro-phenyl)-oxazole

The above prepared 5-(4-fluoro-phenyl)-oxazole (1.82 g, 11.2 mmol) was dissolved in 25 ml of abs. THF, cooled to −78° C., and deprotonated by adding via syringe 7.67 mL of nBuLi (1.6 M (hexane), 1.1 eq.). Hexachloroethane (3.961 g, 1.5 eq.) was added after 0.25 h all at once as solid and the mixture allowed reaching slowly rt. Pouring onto crashed ice/NH₄Cl, twofold extraction with AcOEt, washing with water and brine, drying over magnesium sulfate, and evaporation of the solvents, followed by flash chromatography (SiO₂, hexane/AcOEt=95/5), yielded 1.895 g of the title compound as off-white crystals.

MS (El): 197.1 [M]⁺.

### Step 3: 4-[5-(4-Fluoro-phenyl)-oxazol-2-ylamino]-piperidine-1-carboxylic acid tert-butyl ester

The above prepared 2-chloro-5-(4-fluoro-phenyl)-oxazole (1.00 g, 5.06 mmol) was condensed with 4-amino-piperidine-1-carboxylic acid tert-butyl ester (1.115 g, 1.1 eq.) in the presence of 1.5 eq. of N-ethyldiisopropylamine (1.29 mL) under microwave irradiation in 15 mL of acetonitrile at 175° C. for 2.5 h. Cooling, pouring onto crashed ice/NH₄Cl, twofold extraction with AcOEt, washing with water and brine, drying over magnesium sulfate, and evaporation of the solvents, followed by flash chromatography (SiO₂, hexane/AcOEt=1/1), yielded 1.112 g of the title compound as off-white crystals.

MS (ISP): 362.1 [M+H]⁺.

### Step 4: [5-(4-Fluoro-phenyl)oxazol-2-yl]-piperidin-4-yl-amine hydrochloride

The above prepared 4-[5-(4-fluoro-phenyl)-oxazol-2-ylamino]-piperidine-1-carboxylic acid tert-butyl ester (1.100 g, 3.04 mmol) was treated with 15 mL of 4N HCl in dioxane. After stirring the suspension for 1 h at ambient temperature, TLC indicated the absence of starting material. Evaporation of all volatiles left 1.04 g of the title compound as hydrochloride as off-while solid.

MS (ISP): 262.0 [M+H]⁺.

### Step 5: [1-(2-Ethoxy-4′-fluoro-biphenyl-4-ylmethyl)-piperidin-4-yl]-[5-(4-fluoro-phenyl)-oxazol-2-yl]-amine

The above prepared [5-(4-fluoro-phenyl)-oxazol-2-yl]-piperidin-4-yl-amine (0.100 g, 0.29 mmol, corrected) was dissolved in 3.5 mL of iPrOH and treated successively with 2-ethoxy-4′-fluoro-biphenyl-4-carbaldehyde (0.071 g, 1 eq.), titanium tetra-isopropoxide (0.26 ml, 3 eq.), and N-ethyldiisopropylamine (0.10 mL, 2 eq.), followed by NaCNBH₃ (0.036 g, 2 eq.) after stirring for 0.25 h. The reaction mixture was allowed to react over night and then poured directly onto a flash column (SiO₂). Elution with AcOEt, followed by a second column with the isolated crude product (SiO₂, AcOEt/2% NEt₃) produced finally 0.076 g of the title compound as white crystals.

MS (ISP): 490.1 [M+H]⁺.

### Example 32

### [1-(2,6-Diethoxy-4′-fluoro-biphenyl-4-ylmethyl)-piperidin-4-yl]-[5-(4-fluoro-phenyl)-oxazol-2-yl]-amine

The compound was prepared in analogy to Example 31, but using in the reductive amination step 2,6-diethoxy-4′-fluoro-biphenyl-4-carbaldehyde instead of 2-ethoxy-4′-fluoro-biphenyl-4-carbaldehyde, as white crystals.

MS (ISP): 534.3 [M+H]⁺.

### Example 33

### [1-(2,6-Diethoxy-4′-fluoro-biphenyl-4-ylmethyl)-piperidin-4-yl]-(5-phenyl-oxazol-2-yl)-amine

The compound was prepared in analogy to Example 32, but starting with commercially available 5-phenyl-oxazole, as white solid.

MS (ISP): 516.3 [M+H]⁺.

### Example 34

### [1-(3,5-Diethoxy-4-fluoro-benzyl)-piperidin-4-yl]-(5-phenyl-oxazol-2-yl)-amine

The compound was prepared in analogy to Example 33, but using in the reductive amination step 3,5diethoxy-4-fluoro-benzaldehyde Instead of step 2,6-diethoxy-4′-fluoro-biphenyl-4-carbaldehyde, as off-white solid.

MS (ISP): 440.3 [M+H]⁺.

### Example 35

### [1 -(3,5-Diisopropoxy-benzyl)-piperidin-4-yl]-[5-(4-fluoro-phenyl)-oxazol-2-yl]-amine

The compound was prepared in analogy to Example 32, but using in the reductive amination step 3,5-diisopropoxy-benzaldehyde instead of step 2,6-diethoxy-4′-fluoro-biphenyl-4-carbaldehyde, as white crystals.

MS (ISP): 468.3 [M+H]⁺.

### Example 36

### [1-(3,5-Diethoxy-4-fluoro-benzyl)-piperidin-4-yl]-[5-(4-fluoro-phenyl)-oxazol-2-yl]-amine

The compound was prepared in analogy to Example 32, but using in the reductive amination step 3,5-diethoxy-4-fluoro-benzaldehyde instead of step 2,6-diethoxy-4′-fluoro-biphenyl-4-carbaldehyde, as white crystals.

MS (ISP): 458.2 [M+H]⁺.

### Example 37

### [1-(4-Chloro-3,5-diethoxy-benzyl)-piperidin-4-yl]-(5-phenyl-oxazol-2-yl)amine

The compound was prepared in analogy to Example 33, but using in the reductive amination step 4-chloro-3,5diethoxy-benzaldehyde instead of step 2,6-diethoxy-4′-fluoro-biphenyl-4-carbaldehyde, as white foam.

MS (ISP): 456.3 [M+H]⁺.

### Example 38

### [1-(3-Ethoxy-4-methyl-benzyl)-piperidin-4-yl]-(5-phenyl-oxazol-2-yl)-amine

The compound was prepared in analogy to Example 33, but using in the reductive amination step 3-ethoxy4-methyl-benzaldehyde instead of step 2,6-diethoxy-4′-fluoro-biphenyl-4-carbaldehyde, as white foam.

MS (ISP): 392.0 [M+H]⁺.

### Example 39

### [1-(4-Chloro-3,5-diethoxy-benzyl)-piperidin-4-yl]-[5-(4-fluoro-phenyl)-oxazol-2-yl]-amine

The compound was prepared in analogy to Example 32, but using in the reductive amination step 4-chloro-3,5-diethoxy-benzaldehyde instead of step 2,6-diethoxy-4′-fluoro-biphenyl-4-carbaldehyde, as white foam.

MS (ISP): 474.0 [M+H]⁺.

### Example 40

### [1-(2,6-Diethoxy-4′-fluoro-bipehenyl-4-ylmethyl)-piperidin-4-yl]-[5-(4-methoxy-phenyl)-oxazol-2-yl]-amine

The compound was prepared in analogy to Example 32, but starting the whole reaction sequence with 4-methoxy-benzaldehyde instead of 4-fluorobenzaldehyde, as white crystals.

MS (ISP): 546.4 [M+H]⁺.

### Example 41

### [1-(4-Chloro-3,5-diethoxy-benzyl)-piperidin-4-yl]-[5-(4-methoxy-phenyl)-oxazol-2-yl]-amine

The compound was prepared in analogy to Example 40, but using in the reductive amination step 4-chloro-3,5-diethoxy-benzaldehyde instead of 2,6-diethoxy-4′-fluoro-biphenyl-4-carbaldehyde, as white solid.

MS (ISP): 486.2 [M+H]⁺.

### Example 42

### [1-(3-Ethoxy-4-methyl-benzyl)-piperidin-4-yl]-[5-(4-methoxy-phenyl)-oxazol-2-yl]-amine

The compound was prepared in analogy to Example 40, but using in the reductive amination step 3-ethoxy-4-methyl-benzaldehyde instead of 2,6-diethoxy4′-fluoro-biphenyl-4-carbaldehyde, as white solid.

MS (ISP): 422.1 [M+H]⁺.

### Example 43

### [1-(3,5-Diisopropoxy-benzyl)-pipieridin-4-yl]-[5-(4-methoxy-phenyl)-oxazol-2-yl]-amine

The compound was prepared in analogy to Example 40, but using in the reductive amination step 3,5-diisopropoxy-benzaldehyde instead of 2,6-diethoxy-4′-fluoro-biphenyl4-carbaldehyde, as white foam.

MS (ISP): 480.3 [M+H]⁺.

### Example 44

### [1-(3,5-Diethoxy-4-fluoro-benzyl)-piperidin-4-yl]-[5-(4-methoxy-phenyl-oxazol-2-yl]-amine

The title compound was prepared in analogy to Example 40, but using in the reductive amination step 3,5-diethoxy-4-fluoro-benzaldehyde instead of 2,6-diethoxy-4′-fluoro-biphenyl-4-carbaldehyde, as white crystals.

MS (ISP): 470.13 [M+H]⁺.

### Example 45

### [1-(3-Ethoxy-4-methyl-benzyl)-piperidin-4-yl]-[5-(4fluoro-phenyl-oxazol-2-yl]-amine

This compound was prepared in analogy to Example 32, but using in the reductive amination step 3-ethoxy-4-methyl-benzaldehyde instead of step 2,6-diethoxy-4′-fluoro-biphenyl-4-carbaldehyde, as white crystals.

MS (ISP): 410.3 [M+H]⁺.

### Example 46

### [1-(3,5-Diethoxy-4-methyl-benzyl)-piperidin-4-yl]-(5-phenyl-oxazol-2-yl)-amine

The compound was prepared in analogy to Example 33, but using in the reductive amination step 3,5-diethoxy-4-methyl-benzaldehyde instead of 2,6diethoxy-4′-fluoro-biphenyl-4-carbaldehyde, as white solid.

MS (ISP): 436.3 [M+H]⁺.

### Example 47

### [4-(4-Chloro-phenyl)-oxazol-2-yl]-[1-(3-ethoxy-4-methyl-benzyl)-piperidin-4-yl]-amine

The compound was prepared in analogy to Example 27, but using in the reductive amination step 3-ethoxy-4-methyl-benzaldehyde instead of 4-chloro-3,5-diethoxy-benzaldehyde, as off-white solid.

MS (ISP): 426.1 [M+H]⁺.

### Example 48

### [1-(3-Ethoxy-4-methyl-benzyl)-piperidin-4-yl]-[4-(4-fluoro-phenyl-oxazol-2-yl]-amine

The title compound was prepared in analogy to Example 17, but using in the reductive amination step 3-ethoxy-4-methyl-benzaldehyde instead of 2,6-diethoxy-4′-fluoro-biphenyl-4-carbaldehyde, as white crystals.

MS (ISP): 410.1 [M+H]⁺.

### Example 49

### [1-(2.6-Diethoxy-4′-fluoro-biphenyl-4-ylmethyl)-piperidin-4-yl]-[4-4-trifluoromethyl-phenyl)-oxazol-2-yl]-amine

The compound was prepared in analogy to Example 15, albeit in significantly lower yield, but starting the reaction sequence with 2-bromo-1-(4-trifluoromethyl-phenyl)-ethanone instead of 2-bromo-1-(3-methoxy-phenyl)-ethanone, as colorless semisolid.

MS (ISP): 584.3 [M+H]⁺.

### Example A

Film coated tablets containing the following ingredients can be manufactured in a conventional manner:

| Ingredients | Per tablet | |
|---|---|---|
| Kernel: | | |
| Compound of formula I | 10.0 mg | 200.0 mg |
| Microcrystalline cellulose | 23.5 mg | 43.5 mg |
| Lactose hydrous | 60.0 mg | 70.0 mg |
| Povidone K30 | 12.5 mg | 15.0 mg |
| Sodium starch glycolate | 12.5 mg | 17.0 mg |
| Magnesium stearate | 1.5 mg | 4.5 mg |
| (Kernel Weight) | 120.0 mg | 350.0 mg |
| Film Coat: | | |
| Hydroxypropyl methyl cellulose | 3.5 mg | 7.0 mg |
| Polyethylene glycol 6000 | 0.8 mg | 1.6 mg |
| Talc | 1.3 mg | 2.6 mg |
| Iron oxide (yellow) | 0.8 mg | 1.6 mg |
| Titanium dioxide | 0.8 mg | 1.6 mg |

The active ingredient is sieved and mixed with microcristalline cellulose and the mixture is granulated with a solution of polyvinylpyrrolidone in water. The granulate is mixed with sodium starch glycolate and magnesium stearate and compressed to yield kernels of 120 mg or 350 mg, respectively. The kernels are lacquered with an aqueous solution/suspension of the above mentioned film coat.

### Example B

Capsules containing the following ingredients can be manufactured in a conventional manner:

| Ingredients | Per capsule |
|---|---|
| Compound of formula I | 25.0 mg |
| Lactose | 150.0 mg |
| Maize starch | 20.0 mg |
| Talc | 5.0 mg |

The components are sieved and mixed and filled into capsules of size 2.

### Example C

Injection solutions can have the following composition:

| Compound of formula I | 3.0 | mg |
|---|---|---|
| Gelatine | 150.0 | mg |
| Phenol | 4.7 | mg |
| Sodium carbonate | to obtain a final pH of 7 | |
| Water for injection solutions | ad 1.0 | ml |

### Example D

Soft gelatin capsules containing the following ingredients can be manufactured in a conventional manner:

| | |
|---|---|
| Capsule contents | |
| Compound of formula I | 5.0 mg |
| Yellow wax | 8.0 mg |
| Hydrogenated Soya bean oil | 8.0 mg |
| Partially hydrogenated plant oils | 34.0 mg |
| Soya bean oil | 110.0 mg |
| Weight of capsule contents | 165.0 mg |
| Gelatin capsule | |
| Gelatin | 75.0 mg |
| Glycerol 85% | 32.0 mg |
| Karion 83 | 8.0 mg |
| | (dry matter) |
| Titanium dioxide | 0.4 mg |
| Iron oxide yellow | 1.1 mg |

The active ingredient is dissolved in a warm melting of the other ingredients and the mixture is filled into soft gelatin capsules of appropriate size. The filled soft gelatin capsules are treated according to the usual procedures.

### Example E

Sachets containing the following ingredients can be manufactured in a conventional manner:

| | |
|---|---|
| Compound of formula I | 50.0 mg |
| Lactose, fine powder | 1015.0 mg |
| Microcrystalline cellulose (AVICEL PH 102) | 1400.0 mg |
| Sodium carboxymethyl cellulose | 14.0 mg |
| Polyvinylpyrrolidone K 30 | 10.0 mg |
| Magnesium stearate | 10.0 mg |
| Flavoring additives | 1.0 mg |

The active ingredient is mixed with lactose, microcrystalline cellulose and sodium carboxymethyl cellulose and granulated with a mixture of polyvinylpyrrolidone in water. The granulate is mixed with magnesium stearate and the flavouring additives and filled into sachets.

It is to be understood that the invention is not limited to the particular embodiments of the invention described above, as variations of the particular embodiments may be made and still fall within the scope of the appended claims.

What is claimed is:

1. A compound of formula I, 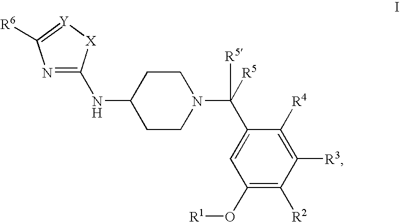
wherein
X is O or NR⁸, wherein R⁸ is hydrogen or C₁₋₇-alkyl;
Y is CR⁷or N;
R¹ is selected from the group consisting of ethyl, 2-fluoroethyl, isopropyl and isobutyl;
R² is selected from the group consisting of hydrogen, C₁₋₇-alkyl,
hydroxy, C₁₋₇-alkoxy,
C₃₋₇-cycloalkyl, —O—C₃₋₇-cycloalkyl,
halogen, halogen-C₁₋₇-alkyl,
—C(O)OR⁹, wherein R⁹ is C₁₋₇-alkyl,
—NH—C(O)—R¹⁰, wherein R¹⁰ is C₁₋₇alkyl, amino,
pyridyl, imidazolyl, triazolyl, pyrrolyl, phenyl, and
phenyl substituted by one to three substituents selected from the group consisting of halogen, halogen-C₁₋₇-alkyl and halogen-C₁₋₇-alkoxy;
R³ is selected from the group consisting of hydrogen, C₁₋₇-alkoxy, amino,
—NH—C(O)—R¹¹, wherein R¹¹ is C₁₋₇-alkyl,
—O-benzyl and —O-tetrahydropyranyl;
or R² and R³ are bonded to each other to form a ring together with the carbon atoms they are attached to and R² and R³ together are
—CH═CH—NH— or —CH═CH—C(CH₃)₂—O—;
R⁴ is selected from the group consisting of hydrogen, halogen, pyridyl and pyrimidyl;
R⁵ and R^{5′} independently from each other are selected from hydrogen or methyl;
one of R⁶ or R⁷ is phenyl or phenyl substituted by one to three substituents selected from the group consisting of
alogen, halogen-C₁₋₇-alkyl, halogen-C₁₋₇-alkoxy,
hydroxy, C₁₋₇-alkoxy, hydroxy-C₁₋₇-alkyl, hydroxy-C₂₋₇-alkoxy,
di-hydroxy-C₃₋₇alkoxy, C₁₋₇-alkoxy-C₂₋₇-alkoxy, C₁₋₇alkoxy-hydroxy-C₃₋₇-alkoxy,
C₃₋₇-cycloalkyl-C₁₋₇-alkoxy, C₃₋₇-cycloalkoxy,
C₁₋₇-alkyl, C₃₋₇-cycloalkyl,
cyano, cyano-C₁₋₇-alkoxy,
C₁₋₇-alkylamino, di-C₁₋₇-alkylamino, amino-C₂₋₇-alkoxy, amino-C₁₋₇-alkyl,
C₁₋₇-alkylsulfonyl, —O—C₁₋₇-alkylsulfonyl, C₁₋₇-alkylsulfonyl-C₂₋₇-alkoxy,
fluorophenyl, pyridyl, tetrazolyl and tetrazolyI-C₁₋₇-alkoxy,
—C₁₋₇-alkyl-C(O)NR¹²R¹³ or —O—C₁₋₇-alkyl-C(O)NR¹²R¹³, wherein R¹¹ and R¹² are C₁₋₇-alkyl,
—C₁₋₇-alkyl-C(O)OR¹⁴, wherein R¹⁴ is C₁₋₇-alkyl, and
—O—C₁₋₇-alkyl-C(O)OR¹⁵, wherein R¹⁵ is C₁₋₇-alkyl,
and the other one of R⁶ or R⁷ is hydrogen or absent in case Y is N;
or R⁶ and R⁷ are bonded to each other to form a ring together with the carbon atoms they are attached to and R⁶ and R⁷ together are —H═CH—CH═CH—;
and pharmaceutically acceptable salts thereof.

2. The compound according to claim 1, wherein R¹ is ethyl or isopropyl.

3. The compound according to claim 1, wherein R² is selected from the group consisting of hydrogen, Cl₁₋₇-alkyl, hydroxy, C₁₋₇-alkoxy, C₃₋₇-cycloalkyl, —O—C₃₋₇-cycloalkyl, halogen, halogen-C₁₋₇-alkyl, amino, phenyl and phenyl substituted by one to three substituents selected from the group consisting of halogen, halogen-C₁₋₇-alkyl and halogen-C₁₋₇-alkoxy.

4. The compound according to claim 1, wherein R² is selected from the group consisting of hydrogen, C₁₋₇-alkyl, hydroxy, C₁₋₇-alkoxy, halogen, amino and phenyl substituted by one to three substituents selected from the group consisting of halogen, halogen-C₁₋₇-alkyl and halogen-C₁₋₇-alkoxy.

5. The compound according to claim 1, wherein R² is selected from the group consisting of halogen, amino and phenyl substituted by halogen.

6. The compound according to claim 1, wherein R² is halogen.

7. The compound according to claim 1, wherein R³ is hydrogen or C₁₋₇-alkoxy.

8. The compound according to claim 1, wherein R³ is C₁₋₇alkoxy.

9. The compound according to claim 1, wherein R² and R³ are bonded to each other to form a ring together with the carbon atoms they are attached to and R² and R³ together are —CH═CH—C(CH₃)₂—O—.

10. The compound according to claim 1, wherein R⁴ is hydrogen.

11. The compound according to claim 1, wherein R⁵ and R5′ are hydrogen.

12. The compound according to claim 1, wherein X is O and Y is N.

13. The compound according to claim 1, wherein R⁶ is phenyl or phenyl substituted by one to three substituents selected from the group consisting of halogen, halogen-C₁₋₇-alkyl, halogen-C₁₋₇-alkoxy, hydroxy, C₁₋₇-alkoxy, hydroxy-C₁₋₇-alkyl, hydroxy-C₃₋₇-alkoxy, di-hydroxy-C₃₋₇-alkoxy, C₁₋₇-alkoxy-C₂₋₇-alkoxy, C₁₋₇-alkoxy-hydroxy-C₃₋₇-alkoxy, C₃₋₇-cycloalkyl-C₁₋₇-alkoxy, C₃₋₇-cycloalkoxy, C₁₋₇-alkyl, C₃₋₇-cycloalkyl, cyano, cyano-C₁₋₇-alkoxy, C₁₋₇-alkylamino, di-C₁₋₇-alkyl-amino, amino-C₂₋₇-alkoxy, amino-C₁₋₇-alkyl, C₁₋₇-alkylsulfonyl, —O—C₁₋₇-alkylsulfonyl, C₁₋₇-alkylsulfonyl-C₂₋₇alkoxy, fluorophenyl, pyridyl, tetrazolyl and tetrazolyl-C₁₋₇-alkoxy, —C₁₋₇-alkyl-C(O)NR¹²R¹³ or —O—C₁₋₇-alkyl-C(O)NR¹²R¹³, wherein R¹¹ and R¹² are C₁₋₇-alkyl, —C₁₋₇-alkyl-C(O)OR¹⁴, wherein R¹⁴ is C₁₋₇-alkyl, and —O—C₁₋₇-alkyl-C(O)OR¹⁵,
wherein R¹⁵ is C₁₋₇-alkyl.

14. The compound according to claim 1, wherein R⁶ is phenyl or phenyl substituted by one to three substituents selected from the group consisting of halogen, halogen-C₁₋₇-alkyl and C₁₋₇-alkoxy.

15. The compound according to claim 1, wherein X is NR⁸ and Y is CR⁷.

16. The compound according to claim 1, wherein R⁸ is hydrogen or C₁₋₇-alkyl and wherein R⁶ and R⁷ are bonded to each other to form a ring together with the carbon atoms they are attached to and R⁶ and R⁷ together are —CH═CH—CH═CH—.

17. The compound according to claim 1, wherein X is O and Y is CR⁷.

18. The compound according to claim 17, wherein one of R⁶ or R⁷ is phenyl or phenyl substituted by one to three substituents selected from the group consisting of halogen, halogen-C₁₋₇-alkyl, halogen-C₁₋₇-alkoxy, hydroxy, C₁₋₇-alkoxy, hydroxy-C₁₋₇-alkyl, hydroxy-C₂₋₇-alkoxy, di-hydroxy-C₃₋₇-alkoxy, C₁₋₇-alkoxy-C₂₋₇-alkoxy, C₁₋₇-alkoxy-hydroxy-C₃₋₇-alkoxy, C₃₋₇-cycloalkyl-C₁₋₇-alkoxy, C₃₋₇-cycloalkoxy, C₁₋₇-alkyl, C₃₋₇-cycloalkyl, cyano, cyano-C₁₋₇-alkoxy, C₁₋₇-alkylamino, di-C₁₋₇-alkyl-amino, amino-C₂₋₇-alkoxy, amino-C₁₋₇-alkyl, C₁₋₇-alkylsulfonyl, —O—C₁₋₇alkylsulfonyl, C₁₋₇-alkylsulfonyl-C₂₋₇-alkoxy, fluorophenyl, pyridyl, tetrazolyl and tetrazolyl-C₁₋₇-alkoxy, —C₁₋₇-alkyl-C(O)NR¹²R¹³ or —O—C₁₋₇-alkyl-C(O)NR¹²R¹³, wherein R¹¹ and R¹² are C₁₋₇-alkyl, —C₁₋₇-alkyl-C(O)OR¹⁴, wherein R¹⁴ is C₁₋₇-alkyl, and —O—C₁₋₇-alkyl-C(O)OR¹⁵, wherein R¹⁵ is C₁₋₇-alkyl,
and the other one of R⁶ or R⁷ is hydrogen.

19. The compound according to claim 17, wherein R⁷ is hydrogen and R⁶ is phenyl or phenyl substituted by one to three substituents selected from the group consisting of halogen, halogen-C₁₋₇-alkyl and C₁₋₇-alkoxy.

20. The compound according to claim 17, wherein R⁶ is hydrogen and R⁷ is phenyl or phenyl substituted by one to three substituents selected from the group consisting of halogen, halogen-C₁₋₇-alkyl and C₁₋₇-alkoxy.

21. The compound according to claim 1, selected trom the group consisting of
[1-(3-ethoxy-4-methyl-benzyl)-piperidin-4-yl]-(3-phenyl-[1,2,4]oxadiazol-5-yl)-amine,
[1-(4-chloro-3-ethoxy-benzyl)-piperidin-4-yl]-(3-phenyl-[1,2,4]oxadiazol-5-yl)-amine,
2-ethoxy-4-[4-(3-phenyl-[1,2,4]oxadiazol-5-ylamino)-piperidin-1-ylmethyl]-phenol,
[1-(3-ethoxy-4-methoxy-benzyl)-piperidin-4-yl]-(3-phenyl-[1,2,4]oxadiazol-5-yl)-amine,
[1-(3-isobutoxy-4-methoxy-benzyl)-piperidin-4-yl]-(3-phenyl-[1,2,4]oxadiazol-5-yl)-amine,
{1-[3-(2-fluoro-ethoxy)-4-methoxy-benzyl]-piperidin-4-yl}-(3-phenyl-[1,2,4]oxadiazol-5-yl)-amine,
1-(8-ethoxy-2,2-dimethyl-2H-chromen-6-ylmethyl)-piperidin-4-yl]-(3-phenyl-[1,2,4]oxadiazol-5-yl)-amine,
[1-(3,5-diethoxy-4-fluoro-benzyl)-piperidin-4-yl]-(3-pheny-[1,2,4]oxadiazol-5-yl)-amine,
[1-(4-amino-3,5-diethoxy-benzyl)-piperidin-4-yl]-(3-phenyl-[1,2,4]oxadiazol-5-yl)-amine,
(1H-benzoimidazol-2-yl)-{1-[3-ethoxy-4-(1-ethyl-propoxy)-benzyl]-piperidin-4-yl}-amine,
[1-(4-chloro-3-ethoxy-benzyl)-piperidin-4-yl]-(1-methyl-1H-benzoimidazol-2-yl)-amine,
[1-(3-ethoxy-4-methoxy-benzyl)-piperidin-4-yl]-(1-methyl-1H-benzoimidazol-2-yl)-amine,
[1-(3,5-diisopropoxy-benzyl)-piperidin-4-yl]-(1-methyl-1H-benzoimidazol-2-yl)-amine,
[1-(3,5-diethoxy-4-fluoro-benzyl)-piperidin-4-yl]-(1-methyl-1H-benzoimidazol-2-yl)-amine,
[1-(2,6-diethoxy-4′-fluoro-biphenyl-4-ylmethyl)-piperidin-4-yl]-[4-(3-methoxy-phenyl)-oxazol-2-yl]-amine,
[1-(3,5-diethoxy-4-fluoro-benzyl)-piperidin-4-yl]-[4-(3-methoxy-phenyl)-oxazol-2-yl]-amine,
[1-(2,6-diethoxy-4′-fluoro-biphenyl-4-ylmethyl)-piperidin-4-yl]-[4-(4-fluoro-phenyl)-oxazol-2-yl]-amine,
[1-(2-ethoxy-4′-fluoro-biphenyl-4-ylmethyl)-piperidin-4-yl]-[4-(4-fluoro-phenyl)-oxazol-2-yl]-amine,
[1-(3,5-diethoxy-4-fluoro-benzyl)-piperidin-4-yl]-[4-(4-fluoro-phenyl)-oxazol-2-yl]-amine,
[1-(4-chloro-3,5-diethoxy-benzyl)-piperidin-4-yl]-[4-(3-methoxy-phenyl)-oxazol-2 -yl]-amine,
[1-(3-ethoxy-4-methyl-benzyl)-piperidin-4-yl]-[4-(3-methoxy-phenyl)-oxazol-2-yl]-amine,
[1-(3,5-diisopropoxy-benzyl)-piperidin-4-yl]-[4-(4-fluoro-phenyl)-oxazol-2-yl]-amine,
[1-(4-chloro-3,5-diethoxy-benzyl)-piperidin-4-yl]-[4-(4-fluoro-phenyl)-oxazol-2-yl]-amine,
[1-(2,6-diethoxy-4′-fluoro-biphenyl-4-ylmethyl)-piperidin-4-yl]-(4-phenyl-oxazol-2-yl)-amine,
[1-(4-chloro-3,5-diethoxy-benzyl)-piperidin-4-yl]-(4-phenyl-oxazol-2-yl)-amine,
[1-(3,5-diisopropoxy-benzyl)-piperidin-4-yl]-(4-phenyl-oxazol-2-yl)-amine,
[1-(4-chloro-3,5-diethoxy-benzyl)-piperidin-4-yl]-[4-(4-chloro-phenyl)-oxazol-2-yl]-amine,
[4-(4-chloro-phenyl)-oxazol-2-yl]-[1-(2,6-diethoxy-4′-fluoro-biphenyl-4-ylmethyl)-piperidin-4-yl]-amine,
[4-(4-chloro-phenyl)-oxazol-2-yl]-[1-(3,5-diethoxy-4-fluoro-benzyl)-piperidin-4-yl]-amine,
[1-(3,5-diethoxy-4-fluoro-benzyl)-piperidin-4-yl]-(4-phenyl-oxazol-2-yl)-amine,
[1-(2-ethoxy-4′-fluoro-biphenyl-4-ylmethyl)-piperidin-4-yl]-[5-(4-fluoro-phenyl)-oxazol-2yl]-amine,
[1-(2,6-diethoxy-440 -fluoro-biphenyl-4-ylmethyl)-piperidin-4-yl]-[5-(4-fluoro-phanyl)-oxazol-2-yl]-amine,
[1-(2,6-diethoxy-4′-fluoro-biphenyl-4-ylmethyl)-piperidin-4-yl]-(5-phenyl-oxazol-2-yl)-amine,
[1-(3,5-diethoxy-4-fluoro-benzyl)-piperidin-4-yl]-(5-pheny,-oxazol-2-yl)-amine,
[1-(3,5-diisopropoxy-benzyl)-piperidin-4-yl]-[5-(4-fluoro-phenyl)-oxazol-2-yl]-amine,
[1-(3,5-diethoxy-4-fluoro-benzyl)-piperidin-4-yl]-[5-(4-fluoro-phenyl)-oxazol-2-yl]-amine,
[1-(4-chloro-3,5-diethoxy-benzyl)-piperidin -4-yl]-(5-phenyl-oxazol-2-yl)-amine,
[1-(3-ethoxy-4-methyl-benzyl)-piperidin-4-yl]-(5-phenyl-oxazol-2-yl)-amine,
[1-(4-chloro-3,5-diehoxy-benzyl)-piperidin-4-yl]-[5-(4-fluoro-phenyl)-oxazol-2-yl]-amine,
[1-(2,6-diethoxy-4′-fluoro-biphenyl-4-ylmethyl)-piperidin-4-yl]-[5-(4 -methoxy-phenyl)-oxazol-2-yl]-amine,
[1-(4-chloro-3,5-diethoxy-benzyl)-piperidin-4-yl]-[5-(4-methoxy-phenyl)-oxazol-2-yl]-amine,
[1-(3-ethoxy-4-methyl-benzyl)-piperidin-4-yl]-[5-(4-methoxy-phenyl)-oxazol-2-yl]-amine,
[1-(3,5-diisopropoxy-benzyl)-piperidin-4-yl]-[5-(4-methoxy-phenyl)-oxazol-2-yl]-amine,
[1-(3,5-diethoxy-4-fluoro-benzyl)-piperidin-4-yl]-[5-(4-methoxy-phenyl)-oxazol-2-yl]-amine,
[1-(3-ethoxy-4-methyl-benzyl)-piperidin-4-yl]-[5-(4-fluoro-phenyl)-oxazol-2-yl]-amine,
[1-(3,5-diethoxy-4-methyl-benzyl)-piperidin-4-yl]-(5-phenyl-oxazol-2-yl)-amine,
[4-(4-chloro-phenyl)-oxazol-2-yl]-[1-(3-ethoxy-4-methyl-benzyl)-piperidin-4-yl]-amine,
[1-(3-ethoxy-4-methyl-benzyl)-piperidin-4-yl]-[4-(4-fluoro-phenyl)-oxazol-2-yl]-amine,
[1-(2,6-diethoxy-4′-fluoro-biphenyl-4-ylmethyl)-piperidin-4-yl]-[4-(4-trifluoromethyl-phenyl)-oxazol-2-yl]-amine,
and pharmaceutically acceptable salts thereof.

22. The compound according to claim 1, selected from the group consisting of
[1-(4-amino-3,5-diethoxy-benzyl)-piperidin-4-yl]-(3-phenyl-[1,2,4]oxadiazol-5-yl)-amine,
[1-(3,5-diisopropoxy-benzyl)-piperidin-4-yl]-(1-methyl-1H-benzoimidazol-2-yl)-amine,
[1-(3,5-diethoxy-4-fluoro-benzyl)-piperidin-4-yl]-(1-methyl-1H-benzoimidazol-2-yl)-amine,
[1-(2,6-diethoxy4′-fluoro-biphenyl-4-ylmethyl)-piperidin-4-yl]-[4-(4-fluoro-phenyl)-oxazol-2-yl]-amine,
[1-(3,5-diethoxy-4-fluoro-benzyl)-piperidin-4-yl]-[4-(4-fluoro-phenyl)-oxazol-2-yl]-amine,
[1-(4-chloro-3,5-diethoxy-benzyl)-piperidin-4-yl]-(4-phenyl-oxazol-2-yl)-amine,
[1-(4-chloro-3,5-diethoxy-benzyl)-piperidin-4-yl]-[4-(4-chloro-phenyl)-oxazol-2-yl]-amine,
[1-(3,5-diethoxy-4-fluoro-benzyl)-piperidin-4-yl]-(4-phenyl-oxazol-2-yl)-amine,
[1-(4-chloro-3,5-diethoxy-benzyl)-piperidin-4-yl]-(5-pheny-oxazol-2-yl)-amine,
and pharmaceutically acceptable salts thereof.

23. A process for the manufacture of compounds according to any one of claims 1 to 22, comprising the steps of:
a) reacting a piperidine of the formula 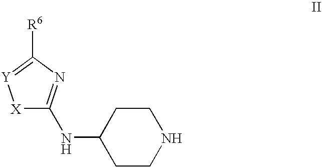
wherein X, Y and R⁶ are as defined in claim 1,
with an aldehyde of the formula 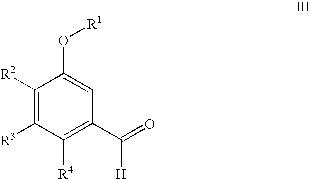
wherein R¹ to R⁴ are as defined in claim 1,
by employing a reducing agent to obtain a compound of the formula 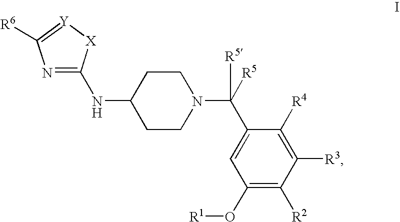
and, if desired, converting the compound of formula I into a pharmaceutically acceptable salt; or, alternatively,
b) alkylating a piperidine of the formula 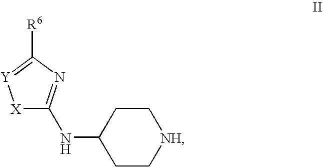
wherein X, Y and R⁶ are as defined in claim 1,
with a compound of the formula 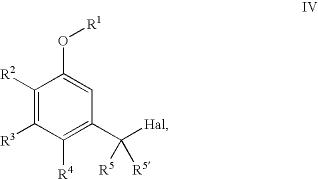
wherein R¹ to R⁵ and R^{5′} are as defined in claim 1 and Hal means a leaving group, under basic conditions to obtain a compound or formula 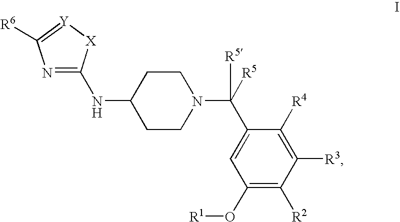
and, if desired, converting the compound of formula I into a pharmaceutically acceptable salt; or, alternatively,
c) coupling an amine of the general formula 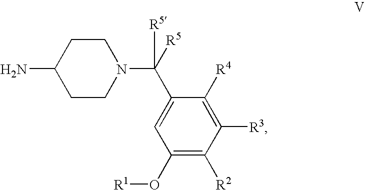
wherein R¹ to R⁵ and R^{5′} are as defined in claim 1,
with a cloride of the formula 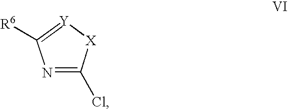
wherein X, Y and R⁶ are as defined herein before, by employing microwave conditions in the presence of a base to obtain a compound of the formula 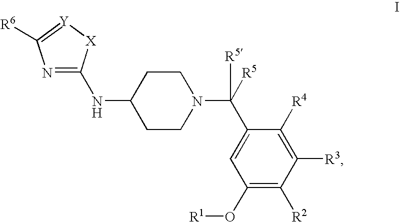
and, if desired, converting the compound of formula I into a pharmaceutically acceptable salt.

24. A pharmaceutical composition, comprising a therapeutically effective amount of a compound according to claim 1 and a pharmaceutically acceptable carrier and/or adjuvant.
